SEQUENCE LISTING (26 sequences)

SEQ ID NO: 1 - DNA, 617 bp, Mus musculus
gccgcagaaagggcagacccgcagcgcgctccatcctttgccctccagtgctgcctttgctccgcaccatgaaccacacttctcaagccttcatcaccgctgccagtggaggacagcccccaaactacgaaagaatcaaggaagaatatgaggtggctgagatgggggcaccgcacggatcggcttctgtcagaactactgtgatcaacatgcccagagaggtgtcggtgcctgaccatgtggtctggtccctgttcaatacactcttcatgaacttctgctgcctgggcttcatagcctatgcctactccgtgaagtctagggatcggaagatggtgggtgatgtgactggagcccaggcctacgcctccactgctaagtgcctgaacatcagcaccttggtcctcagcatcctgatggttgttatcaccattgttagtgtcatcatcattgttcttaacgctcaaaaccttcacacttaatagaggattccgacttccggtcctgaagtgcttcaccctccgcagctgcgtccctccttgcccctccctacacgcaggtgtaacactcatttatctatccacagtggattcaataaagtgcacttgataaccacc

SEQ ID NO: 2 - protein, 137 aa, Mus musculus
MNHTSQAFITAASGGQPPNYERIKEEYEVAEMGAPHGSASVRTTVINMPREVSVPDHVVWSLFNTLFMNFCCLGFIAYAYSVKSRDRKMVGDVTGAQAYASTAKCLNISTLVLSILMVVITIVSVIIIVLNAQNLHT

SEQ ID NO: 3 - DNA, 823 bp, Mus musculus
ggatcacagactgactgctaattgggtcttggttttaggtcttttcaaagactaagcaatcttgttccgagctagcttttgaggcttctgcccatcgcatcgccatggaggaaccatcagagaaagtcgacccaatgaaggaccctgaaactcctcagaagaaagatgaagaggacgctttggatgatacagacgtcctacaaccagaaacactagtaaaggtcatgaaaaagctaaccctaaaccccggtgtcaagcggtccgcacgccggcgcagtctacggaaccgcattgcagccgtacctgtggagaacaagagtgaaaaaatccggagggaagttcaaagcgcctttcccaagagaagggtccgcactttgttgtcggtgctgaaagaccctatagcaaagatgagaagacttgttcggattgagcagagacaaaaaaggctcgaaggaaatgagtttgaacgggacagtgagccattcagatgtctctgcactttctgccattatcaaagatgggatccctctgagaatgcgaaaatcgggaagaattaggagcttacattgtacgctgccctggctgtcgacgatgccgcacagcagatgtgaaagctattttttgtttaagattaaactttttctggtgctgggaaatcttaacttgttaacctttaaattgtagataggatgcacaacgatccagatttatgtgaagtttagaagcctcaagctgtgaggcccagggctgaggaataaagtaaatagaatttggagtatgtacgttctaatttccagaaatttgtaataaaagcatttttgtt

SEQ ID NO: 4 - protein, 150 aa, Mus musculus
MEEPSEKVDPMKDPETPQKKDEEDALDDTDVLQPETLVKVMKKLTLNPGVKRSARRRSLRNRIAAVPVENKSEKIRREVQSAFPKRRVRTLLSVLKDPIAKMRRLVRIEQRQKRLEGNEFERDSEPFRCLCTFCHYQRWDPSENAKIGKN

SEQ ID NO: 5 - DNA, 4925 bp, Rattus sp
cccccccccccccccccccccctccccccccccccacctccgacgtatgatggctcctagacgcaacacgaagcggactccccgcatcattcacgtagacccgccttctgctttccctgtcggggttttgggaagcccggcggccctctcttctcaccttgctccactagcacgcggctgttttcactgagcccagcactggctaagtggagcaccaggagtttcaggctatccttcagagggcaaggtgtagtccatggtgggctacaggagaccctctctctccgtgagtacagagaggcaaacccaagccagacaggggtgatgattaggaacataccttcgtcggggagaaaataccggttcatataggaataagaggaaccaggaggtagttaaggctgtggtgtctggttgcggggtttttgactctcaacaaccacgttcagaacgtgctgagtttttatgatggtgtagaatttccttatcagcaattggtctccgcggtgtttctttttcttttttaattttttaagtataatttggtgtttgaagcaactgtacttggactagaactccctgtgtaatccagaatggaatcccaaatcctaggattaaaggttttagtgggctgcagtgttgggtgggggttgttttgattacgttgtagcccaggctgggctcaatctcaatcctcctgcctctgccttctaaacgctaggattaaaagtgctgcgccatgatcctgctgtagctttatttttatttatttatttatttattttggctctttttttttggagctggggaccgaaccgagggccttgtgcttcctaggcaagcgctctaccactgagctaaatccccaaccccagtgtagctttatttttaagaacaggagtcttgtttctcaaaacagtttctctgtagccctggttgtcctggaactccgtaaaccaggctggtttgggactctgcctttaaaacactgggactaaaggcggtaccacctccgtgggctacaccggaatcttttaagcttcatttgaaccggggctttttctttttctcacccactttctggaagcgattttcctgctaaatttccattcctggtaaatgactctgaggggaaataggaacccagaatagattgagccgggggctacctgggaccccgcactccccaccccccagccgctgttgaagctctttgcctgaggggcctccgggtttgatacctcctagcactccgggctgagggcgtggctcgggaggagccattcctttggagaggaaaacaactgctggccttgaatctgccctaatacctgacagttacatgggacctccttatttccacaggattctttagtctttgtttgggagattttcaaatcttgagactgctcaacccttcctggcctaacactcacaaggccaggctagacccaaattctgtcaaccccttctgtgtccaaaacggtgggtggctagctggctcacccttggtgtcactttgctttaacattcggaaaagttgtggtaagtttcctgtataaaataggaccatctactgggtgtggtcccatgtaaagcaaggttggtttcccaaaataccctgtttacatagatgtccggaagcattggagcaggtcaattagatttaggtggaaacagcctgtttttggaaagctttccagggcggaaaatgaacccagaggcactattgggcaagccctccggctaagcaacacaattggctgcaggggtctctggaagaggtgtgagacaagagagaatatgcaggtttcaggacctctgaactagagttaggctgctgtaacattgtaacattgctgtaagcagaacagcccatggtaagaagctcagtggatctctacaaacactaggatatctgctcagggtttatgaccaggccctgtgcatatggtttgcttcttgttggcccctctcttgaagaggggtgattatctgttacccacttccttgtttctctggggtattaccttgcaaaatgcaaaatgatatacttcactaatgtctccatcttctgtttcagaaatcctacaaccagaaacactagtaaaggtcatgaaaaagctaaccctgaaccccagtgccaagccgacaaaatatcatcgtcgtcaaagggttcgtctccaggttaagagccagcctgtggagaacagaagtgaaagaatcatgagggaagttcaaagcgcctttcccaggagaagggtccgcactctgttgtccgtgctgaaagaccccatagcaaggatgagaagatttgttcgggtgagttgcgtttgtgggcggggcatagatctaagagcaactctagcctcaggaatggcacctaggttaaacagggaatgtagacaaggatagtgactacctgtgattcccagctcaagaaaacaagctccaaggctatcctctactgcgcagtctgaagctggccagagctatatgcaaattgataagtcagtataacatttatttttggattttcagactccctccccatagtccaaactggccctccagttcagtccacggtcctgcttcttccccggtgctaggcttttgagtgataaggctgacttagactggatctcagagctgaagtggacctgttagtctttgtagaccaggctggggtggtttctgctttctcagcgcctagctcacatagtaggcattttaactttgtcttaatagtaatttgagtaattttgtttttctcttgaagattgagcagagacaaagacagcttgaaggaaatgaggtaaatgcatatggatgggtagggtgtctatggatgggtagggtgtcttgtttttactgtttccttagacaaggagtgtgtatgtggagagttaccttctcaacacagggaatctggttattaaagcagtactttaaaaataaataaaataaataaaataaaaataaagcagtagaaggggatttacatttcttttgagttgcaatatcctgattaacatttttctttcagagacgagatgagccattcagatgtctctgcactttctgccattatcagagatgggatccttctgagaatgctaaaatcgggcagaaccagaagaattagggcagtttgaattgtacaccgtccttgccgttaacggtgccatgcagcagatgtgaaagctgtttttttgtttaagattaaacttttcttggtgctggggaaatctcttctaattgctaacctttaaattatataggatgtgtgacatttggattcatgggaatgacagatttacccaagaattgagcatgagtcaaagcctggtagtttgatttagaaggtaattggaataaatctttttattttagattttctagtttgcagagaaatttgtaataaaggcaaatttgttatctttaataaatacagaacagattagaatgagccattggagatgggggactcgttttttacaggtgcatgtgtgggtgtgtgatgttcagagttcaatgtgtgctaccctgtatttctgcttgaggcaaggtctccatgaggcctagctggtctaactcctggtcctgccttttgttttcccctgagttttgacaccataggcttgtcggcaagatctggaagaggcttgatgtttgtgtttgtgctgtgtaataaacaattggttgacatattcctaaagtgtggcactgtattgacctgtctgtctcatgaggaagttaatgaccggagcataattgtatgctttatttcctgagagaagtgtcaggaaaggaggagttaggaagaaagccccaggctggggttaagagcactggctgcttttccagaggtcctgagttcaattcccagcaatcacctggtggctcccgaacatctgtaacaggatccaatgccctcttttggtgtgtctaagaactccctaggcatgcagaggatttttgtttttgtttttttttttttttttttttttttcgtttttttcagagctggggaaccgaacccagggccttgcgcttgctaagcaagcgctctaccactgagctaaatccccaacccctacaatggcctttttctacctgcttttgaattatcaataaaagactggggcaaaagaaaggctggagtgaatgagagagaacatgtgaagagtaaatgagagagagcatgagggaatgaatgagagagtgaatgtgagaacgaatgtgagagcgagtgagagaacatgagaagaacacgttaagagtgagtgaagagagaatgtgaggtgtgtatgaagattgtgtgtggggttggggatttagctcagtggtagagtgcttgcctaggaagcacaaggccctgggttcggtccccagctccaaaaaaaagacccaaaaaaaaaaaaaaaaaaaaagattgtgtgtgtgtgtgaaaggagagtgcatgtggtgtgtgtgagatatgtgcaaggtgtgtatcaagagtgtgtgtgagagtgaaagggtaatgaacagaggtgtgcatgagcgtgggagtttgagaaaagaaaacagcaataaaaaaaaaagcagagtgcacgagagaatgcagagtgtgtgcaacctcaagctgagacagagacagagagaaagagagagagagagagagactttaagccttgaaattacctgtcagtttgtacccaaatagtagtctgtgtatatttattttgagccttccagatccctgcttccagtggagaactctgattctatgttgaggctggaccctggcaatagtgggcttcttgaaaaatagtcaaaggaaacagtgctacaccatggacttaagcctttagactcagttctggcttcaagagcagctgtcagaaaataagtgatgaactacttgcagtcgaactcgaatc

SEQ ID NO: 6 - DNA, 1444 bp, Rattus sp
ccaggattcagacgagctaggcctcatgcatggagaccttgcctcaagcagaaataaacagggtagcacacattgaactctgaacatcacgagtgtgcacacacccacacatgcatctgtaaaaaacgagtccccatctccaatggctcgttctaatctgttctgtgtatttattaaagataacaaatttgcctctattacaaatttctctgcaaactagaaaatctaaaataaaagatctattccaattaccttctaaatcaaactaccgggctttgactcatgctcaattcttgggtaaatctgtcattcccatgaatccaaatgtcacacatcctatataatttaaaggttagcaagtagagatttccccagcaccaagaaaagtttaatcttaaacaaaaaaacagctttcacatctgctgcatggcaccgttaacggcaaggacagtgtatgattcaaactgccctaattcttctggttctgcccaattttagcattctcagaaggatcccatctctgataatggcagaaagtacagagacatctgaatggctcaactcttctctcatttccttcaagctgtctttgtctctgctcaatccgaacaaatcttctcatccttgctatggggtctttcagcaccgacaacagtgtgcggacccttctcttgggaaaggcgctttgaactcccctcatgattctttcacttctgttctccacaggctggctcttaatctggagacgaaccctttgacgaagatgatattttggccgattgagatagaatatcaaaacaacatttaacatttaaataacttaacgatatacacaccttttttttttccacctccccacacagacaaaaaacaaccctattttttctttacaaccccgcctaagcaagcgaagcattagtaactgaccaatcatagaaaggaaacaccaccagaccacatcaaataaaataaaatcaccgcccaaccccacccctataaaaaacccgccgaccacaccacatatactcccccccccccgcaccatcactacatcaccctctccacccattcccacctccccccccaacattaaccccaccccatcacggaaacccccaacaccaacaaataaattagacacatcgcattacataaattgacacaagacccaccccaaaagagcagcaaagattagagccacatcctcggcccaacacaatacactcaacctgcatagtatctatctccaccccaacctagaaacaaaaatctaatcagcaccaggcacccaagtatcacgcacactcaaaaacatacccaccaattaaacacgccccacccacccaacaacccacccgcctgacaacacacttcggaactaccctcaacatcaccaaaagcaatcgcaagttacgatgactccaaccacctcactctctcattg

SEQ ID NO: 7 - DNA, 7656 bp, Rattus sp
  misc_feature (7471)..(7471): “n” is an unknown nucleotide
ctgcaagtagttcatcatttacagatcaaaagaaagaagaataaaaaaacaaggtgtcatgatccctccaaaagagtggaacacttcaactgccagatccaagatactgaaatgggtagcatgctggagaaagaattcaaaagttaggtagagaatctggttgagcagagcacttgcttttcttccagaggatctgagttcaagtcccaggacctatatcacagttttctgtaactctagctccagagggtctgacacttctgttcactgtgggcacctgcattcacagacaaacataaagtagttcatcacccttttcacagaaaacccacagcatgtgaggaaatccgggtctctgcgcaatgcccccacagcagaaggggggagctggagagatggttcatctgttagcccatttattgctcttgaagagaacccagggtcatccatagcacccatagcagctcacaaccatctccagttccaggagatccaatgccctgttgtgacctcaggtaccaggcatacacaatgaacctgcacacatacaaaagtccatagagccatagttaccattgtgagctctgagaaccaaatccgtgttctctgcaagagcgacatgcacgctgagaaccaggcacctttcccactgcctcttgagacaagatctcactatgtagttcacactggcttccgacttgccaccatcctcctgcctctgcctataaagaatgctaggattatataggtacaaaatcacacctggctgttaaggtttttctggctgttttttttttcacccccatgaatgattttgaaaatagttgagctgtttacattaataaaacaaaatcagatggagactatatgtcattattcatgaatcaaatgactagtaacaatactgagttatttttatagcttttctatttttgttttaaattttattttttccttttttttttttttctttttagttttgctttgttttgttttgagcaggctctcactgtgtagtcctgggtgatctggaacttactaggtaaacaaggatagccttaaactcaagaaatttgcttgcctctgtctccagagtgctgcagttaaagttgtacaccgccatgtttaggtgtttttattagtgtgtgtgtatgtctgtgtgtctgtgtgtgtgtgtgtgttccccggaggccatgtaggcgcatgcttgaaccagaaccagaggaagtgtgtttacagttaccctgggaggccagaagagggcaggagatgccctggaactggaatttctggtagtggttaactgcctaaagtgctgggacctaacactcttaacttctgagccatggctctagtcctggggtcccccctccttctttttatgactatgcagactatacaaatttattttatatattaaggtctacgggagcagtttgccctggcagagagtatatatatctcatggtgacatacatatctcatggtgacacacatatctcatggtgacacacatatctcatggtgacatacatatctcatggtgacatacatatcatctcatggtgacacaattgagcattgagagcagctacagaccgattagatcagacttattaaattcttgccaagtatgtggtgacgcaggcctgcaatgccagtaactttggagactgagccaagcagatcacctgagcctagagactcaaggccaccctggacaacatagagatatcctgtttcaaaatgaaacaagctaagttctttgtacatagcagcctctctattgactgtggcagggcagctgacagtgttctcacctagtcacagatgttctttctagagggaacagacccgatgaatacaaacatttttagctcaagtaaaagtctatactatgaaggaactacttcttcaaacatcataacatttaaaatgagagattttacaaacctttttttaaagatttatttgtttatgataagtacactgtcactgtcttcagacacaccagaattgggcatcagatctcattacagatggttgtgagccaccatgtggttgttgggaattgaactcaggacctctggaaggacagtcagcactcttttttttttttttttttttctttcattttttcggagctggggaccgaacccagggccttgtgcttgctaggcaagcgctctaccactgagctaaatccccaacccccagccagtgctcttaactgctgagccatcttcccagccccaacatcaatttttggtctagatgttttaccctggtgctgccatgccatctcgatggcccttgtggcaggggtgccggtaaggcagcccctagggcatgagttagggagagcaaaacctgacccagaacctgactgccatgaagtgatggagatgccgtttgagtacatggggttttttggtggttgttgttttgttttgttttttgtttttgttgacttgacacatgctacagtcatctgagagtgaaacttaattgagaaaatgcctctgtattttctccggccccctaagttgcttttgatgagtgtatttttatcacagcaatagaaactctaactaagatagattggtattagaagtagaatattgctgtaacagaccctaaccatgttctcttggggaggattgtgggaagactttggaacttggaacttggaacaggagaagccattgggtacttagagcttaatgggctgttctgtggagcttggaaaggtgctggagaaatgcggatgatacttgtaaagtttgagagcacctcaaagatgttcaggacagtgtgtgcaatacatttgagttaagaatctatggtgtctggtcagctggagctgaagattcagctgtgattaataagaccactaaagtaaaacttttgctttactggtacaatcagtgctggttagctaagggttgacagatgagcagtgactaataagagactggcatcagaaactgatccagagagagccaaggctgcatctcaaactggcagccaaatttgatcacatgtaagaatctccctcatgggggttggggatttagctcagtggtagagcgcttgcctaggaagcacaaggtcctgggttcggtccccagctccgaaaaaaaaagaacaaaaaaaaaaaaaaaaaagaatctccctcatgttacaggctttggtggcatgagagctttagggttgaaggatcatggagagcagccgaggctccgcaccatgtggcggggcagaggtacagcccagttaccacagagacaccagcatatttggaggtgccaggatcatggataattgcctaagacaggaggctggcctgactttgtaggacaagctccatgatctgtttggcaggactggagaaacagagctgtaagggaaaatgaggacacagctgttccaagatatgattggagagaagggtttcattgcagatctgaggaagaggacagccagagaggcatctggaagggtccagattgaactgggtcatgagaggagagagggctaagaggaccaaaagagcctgtgaccaaattatcagggttatagagaaaacagatgcttgggaaagagaagggggagcccctgagctggagagatttaaagtagggggcaggatgagaagtggctggggcaggatgagaagtgctgaggagccaaaggcactcagtgaacctagaggccaaggatacattttgacatgctaataggcattttagtcatttgtcctgcatttctttaggacaggccaagctgcctgggtcattgtgagtcccagataattctcttgaaataaaatgttttttaaagagaggaggggaaggttggggagggtggtctgaagttaagagactttggagtattaagacattggatattttagagaaaattttgaacttttaagaagactgaccttttaaagtgtttgaatttttaaagaccaggatacatcagggtgtagggacacatgaccctgtctcgccccccccccccaaaattataatttttttaaaaagactgtgggagctgggtggtggtataggcctttaatcctagcacccaggaggcagaagcaggcagatctctgagtttgagaccagcctgatctatagcatgatttccaggacaatcaaggctacacagtgaagcctatcttagaaaaaaaaagattgtagttttagtttgcgatgtattttatattgaggtgctgacattaatatgaaatctttgtgagtgggcaagaaaataaagactaaagctgaatactgatgccacttgtgtgtcagattgacaaggggttttggaatttttttatttttttatttttttttaggaatatatcaaccaattgtttattacacagcatgaacaaacacaaaaatcaagccttttccagatcttgctgacaagcctatggtgtcaaaactcggaaacgagaggcaggaccaggagttaaaagaccagcgaggcctcatggagaccttgtctcaagcagaaataaacagggttggtagcacacacgaactctgaacatcacgagtgtgcacatacccacacatgcacctgtaaaaacaaatcccccatctccaatgtctcgttctaatctgttcttgtatttattaaagataacaaatttgcctttattacaaatttctctgcaaactagaaaatctgaaagatctattccaattaccttctaaatcaaactaccaggctttgactcatgctcaattcttgggtaaatttgtcattcgcatgaatccaaatgtcacacatcctatataatttaaaggttaacaagtagaagagatgtccctagcaccaagaaaagtttaatcttaacagaaaacagctttcacatctgctgtgtggcacctttaacggcaaggacggcgtacaattcgaactgccctaattcttctggttctgcccgattttagcattctcagacggatcccatctctgataatggcagaaagtgcagagacatctaaatggctcatctctgttctcatttccttcaagctgtctttgtctctgctcaatccgaacaaatcttctcatccttgctacaggttctttcagcaccgacgacaacaatgtgtggacccttctcttgggaaaggcgctttgaacttccctcatgattctttcacttctgttctccacaggctggttctgaacccggtgacgaaggctgtgatgacgatgatattttggccacttggcactggggttcagggttagctttttcatgacctttactagtgtttctggttgtagggtttctgaatcattggggtgagtcctctccacctttcctctgagatctatcatctgagtttctggatacacaactgggtcaactttctgtgatggctcgtccatggcggtgggcagaagcctcaaaagccagctccgaacaaaattgctagctaatctttggaaagacctagactttggccccaactagcagactgaagtgctggaatttttttttttttttttttttttttttgtaatcaacttgaaaacacaattgagaaaatgcttccataaggttaaatccttgtgccaccatgcctggacctaagcttttcatggccactattcctcgaggtctggatcagaagcttgtgtatttcatttccggattgtcgttcactccagattaaaagtccaaatgaaagcaatagccatgtaataatgcctagatataactcttccttgttcagcagcaaatgcataagcaataagcttagctgggtgggatcttccaaagctactctgctctttttcttcttggacataggattcagcaacattctacttcttgatgcccctttattctttgaaccatacatttttacttttcctttcgtagcttcttccttttcatcaaaagattcttcataagagtgaaatttggggttagagagatggttcagtggttaatagcactgactgctcttccagaggtcctgaattcaattcctagcaaccacatggtagctcataaccatctgtaataggatctgatgccctcttttggtgtgtctgaagaagacagcaacagtactcaacatacataaaataaaaataaatcaacatacataaaataaaaataatttttaaaaaaaaaaggtgaaatttaaccacacaacagaatttatgccaggcttgtttgagacttttgtcaaagcaattaatctaaatctcttcaccttagcctcaggtagactctctggacaatggcaaaaagcagccacattcttcatcaaaatattacaagaacggtctctcagccacatactaaaattcttctctgaaacttctagagccaggcttccacagttcaaaccaccttcagcaacaaagtcttctatattcctacgatgatagccctttaagccccacttaaagcatttcactgaattccaaatctaaagtctccaaatctatattcttccaaataaaagcatggtcagacctacctatcacagcaatatcccagtccctggtaccaacctctgtcttagttagggtttccattgttgtgaagagacaccatgaccaaagaaacactttttttttttttaatatttattttatgtctatgagtacactgttgctgtcttcagacacaccagaagagggcatcagatctcattacaaatggctgtgagccactacgtagttgctgggaattgaactcaggacctctggaagagcagccagtgctcttaaccgccgagccattttctccagtcccaaagaaacacttataaaggacaatgttttttttggttttttttaaaggtttatttattttatgtatatgagtacactgtagctgtcttcagatacaccagaagagggcatcagatcttactatagatggttgtgaaccaccatgtggttgctggggattgaactcaggacctctggaagagcagtcagtgctcttaaccccttagccatctctccagttctaaaggacaatgtttaatcggggctggctcacaggttcagaggttcagtccattatcattgagacaggagcgtggcagcatccaggcaggtgtggggctgaaggagctgaaagttctacctcttgatccaaaggcagaccaaaaaaaagactggcttacgggcttaccataagcagctaagaggaaggtctcaaagcccaccctacagtggcatgttctccaacaaggccacatctcctaatagtgccactccccgggccatgcatattcaagtcgccacacccactgagccatctctccaacctgctccagaccatctcccctgcttttacctaagctcattaggcagcaatatgcctcttattgtttgagctcagcatcctgtttttcaaaaggctgcttgtcatcacagtggtttgttccacaactctcccagtttctttgtnaaaacaccaatgcctagagagatgctcttctgtacatatcgcatgtgcagaagaaagggtgccagatcctttcatgtggaccntgtcatgtctttacccacgtagtcgtctgctctgactcttctcgagatgctganaactgattgagcgtaggatgctctgggtatgtgcatgggacaattttg

SEQ ID NO: 8 - DNA, 2161 bp, Rattus sp
  misc_feature (2115)..(2115): “n” is an unknown nucleotide
cgaaggacggtaaggagagaagaggggagaggatcaggactgaggggagatatgcactgaacgggggagttagtaacgaggaaaagatagggagaaaagtgggagaaaaaaggccggggagggggagggcatggaaagaaaggcgggggggggagataacatgcgggggaagtaagagggggggggtaaggagggtacaggtagcacaggtggggggaagagaggggagggggggaatgggaaaggtgagggtgggtgggggagttttcggcgaaaggggccggagtgtggattatcgcgtggaccagaacgggggaagggccacatttgggtgggcgggaacagaaaggaaatctttttaaatcggttgggtcgcagggtgggtggacattgagaaaaaaatcatcaaagcccctaaggagcatttgtttcggagttatacgtatggatattttattatatgggacgagagataaagaatacttcttaagtaatccctttaaaaataatgtcaggctggagaaatggtttcatgggtaagcaagtgtgagagatgagcgcagacccccaggacctgtgtagacttaatgcagaggtggatgcacgcctgtaatctcagcatgcctacagccagataggagatggggacagagaagtgtgggggccaactagcctggtgtctacagcctggtgtcaacagcagcctcctacctcaaacaaggtggaaggtaagggctgatacctgagatcgttgtctgacctccacacacattgtgcttatactttacacacatactcacactcacacatacatacacatatatacctggtctccattaggcttctattgctgtgataaagattacgaccgaggtctttccaaagactaagcagttttgtttgcagctagtttttgaggcttctgcccaccaccatggaggagccattagagaaatcgacccagttgtggacccagaaactcctcagacgaaagatgaaaaggacgcatccgctgattcagaagtcgtaagccagaaacactagtaaaggtcatgaaaacgctagccctgaaccccagtgccaagcggtcagcacatcgtcgcagcctccgtctccggattcagagaagacctgtggagaacagaagtgaaagaatttcgagggaagttcaaagcgctttacccaagagaagggtccgcacgttgttgtcggtgctgagagatcctatagcaaggatgagaagacttgttgggattgagcagagacaacacaggctggaaggaaatgagtagaaacggaagagtgtgccattcagactcactgtgctttctgccattatcagagacgggatccgtctgagaacgctaaaatcgggaagcattaggacagcttagattgtacactgtccttgtgttaatgatgccatgcagcagacctgaaagctggcttttgctttttaagattaaccttttcctggtgctggggactcttctaacttgttaacctttaaattatatagggtgcgtgatgtttggattcatgtgaatgacttaaatttacccaaagaattgagaaggagtcaaagcattctgtgaatttttgaagcctcaagcccggggccgagaaacaatgttaatagaatttggaatagtttggtttagaaggtaattgggatagatctctgaattttctagtttgcaaaaacaaaaacaaaaaaaaagactaaaaaaacaactggggaggagtaaggttatttcagcctccatgtcttgatcccagtccatcatgaaaggaagtcaggacaggaactcaagtcaggaccgtggaagtaggtagcatctgaagcagagacttctgggatgaaagcgctgcttcctgactcgctccccacaaattggtccctgagccttcttgtccaccctcggaccccttgcctagggttggcaccacccacaatgggctgagccttcccatgtcaatcactaattaagaaaatgctgtacagcgttgcctacaaaccagtcttaaggaggcgttttctccattgtggctctctcttctctgataactctagcttgtgtcaaattgacaaccaaccagccagcacacaaacanttaaaaagatagaaataatgttagtgnntcncatcgagcaagagtc

SEQ ID NO: 9 - DNA, 21688 bp, Rattus sp
tttatgattttaaaagtttaattctggactggagaaatggctcagtggttaagagtagtaactgctcttccagaggtcctgagttcaagtcccagcaaccacatggtggctcacaaccatctgtaatgagatctgatgccctcttctggtgtgtgaagacagctacagtgtattcacatacataaaataaataagtaagtctttaaaaaaaaagtttaattgtgtgtgtgtgtgtgtgtgtgtgtgtgtgtaagcttgcaaataagaggacaactttgaggagctgatactcttgttctactgtgtagggaccaacagttgaactcaggttgtccggcttatgcaacaagcttttttacttgtcttcgccagcccaccagtcctgtgtaaagctgcatacagctcacgttgtaacatgcttgtctagtacttgcaggacataaactagcaagcacttgggtgaaaacgggaggatcagaagttcaatactatccttggctacttaacaagtttaaggctataggaatagggatataggaaaccctaagaaagtaaaatttatttactgtgctttaggtgatcaaacctacagctttgcatgtgatagacaaatgttctaccactaagctacatcctcagtgttctttattatctatttttttaataaatctttttttttaaacattgttgtgagccaccgtgtggttgctgagaattgaactcgggacctctggaaaagcagtcaaggaagccagagtggccggaactcctgaaaatggagtaacaacaggttgttgtgagggtaattgaactcaggtcctatgcaagagcaacaagaggtcttagccctttattattttttaatatctaattatttttttatttttttatttttatttatttattatatataagtacactgtagctgtcttcagatacaccagaagagggcatcagatctctttacagatggttgtgagccaccatgtggttgctgggaattgaactcatgacctctggaagagcagtcgggtgctcttaaccactgagccatctctccagccctaattatttattttatgtatgtgagtacactgtagttgtcttaagacacaccagaagagggcatcgggtatcagatcaccattacagatggttgtgagccaccatgtggttgctgggaattgaactcaggacctctgaagagcagtcagcattcttaacgactgagccatctctccagcccaacccccccctccattttttttaataccaaaaaggagcttcctgcaagagaacatggccatatacatccacccctctttctttgaggttttgatagtgctgctgctcctgctgcttggaaaagaaaatcctctaggactaagctaaaagagccagatggatggaattgcggttgccatggcaacaccatctgaggatactgagcctgctgtctctcccagttatgttgacatttggtgtggtttccatgcttgaacactgaagtgtctgtccacctatgaaagagaggccgttcccagaggtcttaatttatctgctccatcagtagcatttggactgcttacatttatgtctggacaaccattggccaggaggtagaagaggatggaggaaggcccagacctggctgggtactatcggatctagtgaagctgtatagaatctgtctggggtttatttactcccaactggagcagaggcaggtgctcaggaaggcagtaatgagatcgaccttaccacaggaaataaagtgactactgtggataccatctgggatggatcaccgctgagccactccaccctcagaacaaagctaccatatcgttaaagtgtcctgagctcaggggaaggcccctgctgcctgtgagtagagccaggtaaccttaacaagccctatctacacttcatcttaaggcattctgttacatacaaagaattctactctttaatgagcagactttaaaaaaaatgagccaacttacactttcagaagtttgatccttgattgcacatgcctgagacagatggccagtctcaaggacaggcctcccacactgaagttagtcttcagcagtatgtcatgtcacctaggcaaccaataagagctcacctaagaaatttccactttacctggtaaagagcgtatcttccctccctttctctccaattagcatcctcacttccagacttccctactaccgactttaaaagatcaaagccaggcacgatagcacaggctgaggtcggaaggcagaagccagaaagatctatgtgattcccaggctacttagcaccacacagttgagaccctgtctaacaaatggaggtgggaggcatggcagtaacctgaacctacaaatttatcaaaatttcaattaagaacattttgttttgtttttgaggcagaatctcactacgtagagtgggcttacacccagttccaattaagaacattttaagggctggagagatggctcagctgttaagagcactggccactcttcccaaggtcctgagtacaattcccagcaaccacatgatggctcacaaccatctgtaatgaggcctgatgccctcttctcttgtgtctgaagacagctacagtgtcctcatttaaataaaaaaacattttaaatagaaaatccaacagggaggctgatgagaaacgacataacctttgtccaggagtgtggttaaggggaatggaaccatagtagagtccatttctttttctcttttgagccaaaaaagttttatttattcatgtcttccatttgaagtactccttggtggcatcctaagcctgagattctttgccatacgtagttcttaaccactacccaactgcaaccaactgttttctgtggcatccctcttgatgacttttacacaggggttggggatttagctcagtggtagagcgcttgcctaggaagcacaaggccctgggttcggtccccagctccggaaaaaaaaaagatttttacacgggcacacccactccactagtttctcatgatcaagtataatcagattgatctggtgctcggcacaaagtgcctcctccagctcgacacacacgagctcatcacagtcggattcgagcacacagatgggtttggcacttgtctaaggcttcaggagctttgtgtttgccaacgtgctgggctatcgtggatgagggcggtcttcagcacctcttgtagagcagtgttgacatccacacctccagtggcagtgccctgctccgctctcggaagctgaggtggaatagcaagtcagtttcttctctcatttcccagacaccattatggatgcctcagtgtcagctgttcatttgtcacttacttttcacaattgtgttattattattgatagattattgtctctgtcactagctaccgaggcagggtctcacaggacttatccaattgtttctgcctccctcgagctaagcctgaaggcatatatgaatcatctcaccaagcagcatcagcttttaagagtttctgaacgtcaacacgttaacactggggccatattatgtacgatgtaattaatcctcgagcaactggccacacagccctaaaagaaaaaaaaatccagaaccaaacaaaccaaaaacaggcacgaatggtggcacacaccttcaatctttacacttggaaggtggatccaggaggagtaggaattcgaagccggcctagagtaccagtagttgaaggccagcatctgtctcaaagcaaacaacgataataaagtacttgtttcagctgggaggtggtggtacattgtggagggagaggcagaccttgaacactgggttcaaggccagcctggtctagagatcagatccccaaaacagccagggatagacagagaagccctgtctcaaaacgtgaggctggagagatggcttagtggttaagagcactgactgctcttctagagatcctgagttcaattcccagcagctatatggtggctcacaaccatctgtaatgggatctgatgccctcttctgtgtgtctgaagacagctacagtgtacttatatacatgaaataaatctaaaaataataataacgtgcacaatgttctgcctgcctatatgcctgcaagccatccctccaacccaataaataaatattaaaaaaaaaaaaaagcacaaaaccaaaccaaaagtaaaataaataaacaacttttattcctaccaagagaagacacatttccttgagaactaaggacaacatgtttatggttagaacacagaagagaataagagcacagctcagctggaagaaacaaagtgttctggggacaaggagccttcttccctgcccccataacagtggccagattgaacctctggtacgacagtcaagttggtgctgagttcaagttggaaagtcacactttctaaatcaggatcaaagcaagctggaggctccctcactcagctcacaagtcctgtgaaatcaggaaaaaaatatcagttagacactgagttcccaggcagccaaaaaccaaagatttcccaccaccaaagacaaggtatcttggatttccaagggaacagaatgagaacttatatctctgactggcatttaaatcctacagccatcccctctccagcacatcctttctccagggaatggtcccagcacccatgtcaggcactcacccaagtagtcatccatcagagagccaatagcaaactgcgagaggaaagggagaaaggatggtgaggtggggccccaccccattccgagccttctgtcatctattccctgctcatggacacagagcacagagcccccaacaactgtggatggcaagaggtcaacagcgcagatggggaaagagcttgctccaaccctgatgacctgacctccacccccaaaatccacagcagcatgcgatgacctgaaggcggtctaaatgtcacactgtggcgagtgtgtatgcccacacatccacataaatatgttctacaaaagaaacgagaaacccacagctgtcagctgtgaatgatgactttggattatttataatcctactacccaggaggctaaggcaggccagtcaagcaagagactcacaatgtcattcttgtctacacgtgtccctacaatcttcaagcgtatctcatcgtcctgctgaattacaatgtcctgtggaaaggagagagcagggtcatcaagcagactcaggcctggtcctcatccctctcaccaactcctcctcattcgctcacctcatccatggtcttgtaacaaggggggttcgaatttggatcaaactccatctctgaagggatggactagaaggaaattgacacaaaggttagcatttcaaatagctgcatcaaaggatgagagtcaggggctggtttctcctcctcggcctcaccccacacgcccagactcacgtgtcgagagatgaagcaggacatgggcccaatttctgtgaaaagtccaacctagaaggaaaatgaccgtgcttcaaacgctctgaagcatctttacctgatttctaggcacattattcatgtttcttaacagtttaaattgtagcatttgttttaatttctctctgtgtaatctttcatttctttacatttttgttcttcattatttttatgtgtaagaatattctgacctcacatgtgcctgtgcaccatgtacctgcagtgcccatggaagccaggagagggtattgggaccctgcagaattaggagttacagattattgtgagccattggctgggtgctgggagtcaaacccaggtcttatagaaccagtaggtgctctaaaccactgagctatagaccccttagcctttaagaaacttaatttctgaggctagagagatagctcagtggttaagagcactgactgctcttccatgggtcctgagttcaattcccagcaaccacatggtggctcacaaccatctgtaatgagatctgatgccctcttctggtgtgtctgaagagagctacagaggagtgtgtataataaataaatcaggggctagagagatggctcagcggttaagagcactgattgctcttccaatgatcatgagttcaattctcagcaatcacatagtggctcataatcatctgtaatgggatctgatgccctcttctgatgtgtctgaagacaacagtgtactcatataaataaaaataaacaaacaaaccttaaaaaaaaaaaaaaaaaaaagaaaagaaaacccaaaactaagataaaataaaataaatcttgacaaccacaaaaggcttaaggcaactaataagtggactgggaattgaactctcaccttaggaaataccccgtaacctttcttttttttttttttttttttcttcttttttttcggagctgaggaccgaacccaggaccttgcacttcctaggcaagcgctctaccactgagccaaatccccaaccccataacctttctataaataatactcttaccttgttgacctgagtgaccacagcatccaccacttcccctttaaagggccggaaaacaatagctttgtatttcactggataaagaacaaaacctcggcccggctggatcacaccagcaccaatattgtcgatggtagtgacagcaatcacaaagccatatctgcaggaaagatgaaaaaagacagctactgtatgtgaagagcctctaaaaagccaccagcaatagtctgcgtgtgatggaacctctgctcgaacagctcgatgaccaagaagagacagaactcagattagcacctgaaatattaaatggtgctctcacaattgtacagtaaatgcccaagaaggcacagatatgctgacatacacctattctctcagtaccaggacttgccaggtcagtggtgagacaggtctttcgaaaaccacaaatcagacagaaaattgtgacgaaaacctttaatcccagcactcagtggcaggcagttctctgaattagaggccagcttggtccacatagtgaggccatctcgaaacccaaaacatttgcataataacggtctgatctcgcataagcgaagaaaatttggtttagcaaccttttagaaggcccaaaataggcaaaaactggctgcttcggatgcctggagtggtgaaagagttcctcagagtaagtaacaagccctgactgaaggagtgaagtagaggttacagagtagcgttattgtgcctgcattcagcagacgacactgtgaatcagacacttacttcccagtgcaggtcccctccacctcggtgaacagcttctgcttcaccgtgttgagcaagttgggaccaaagtagcgtgggtgcagtaggatctcgtgctccagggaaatctgcagagaaaggaagatgaagactccgccagccacactgagaacaggaggcgacccgtcggccctccaggctcctcctgtccctgccctcaccgctaccccgcgtccagctcacatgataaaacatcttctgcagaagcttggaccgcagaggccagaactccccaggaagggacctcgccggaagcactagcagaagtcccaccaagtctccgcagtcgcttccgcagatttgagtcttaacgccatgggcggggaaacgtgaagccccgcccctcaggccttcccatcagcgctcatcagcacagccaggattacacagaaaaacccggtctcgaaaaaccttaaaaaaaaaaaaaaaaaaaaaaaaaaaaggttaagaggtctggcttgtcgccacatgcctttaaacccagccgtggcagacagatctctaaattcaaggctaagccacatctacaaagtgagttccaggataaccaagactgtgtatacaaaccctataaaaaaatttgtttttggggttggggatttggctcagaggtagagcgcttgcctagcaaccgcaaggccctgggttcggtccccagctccgaaaaagagaaaaaaaaattgttttttaaattttattttaggggctgaagaattagctcagtccttaagagcacttgccagcccccacaggatagctcacaatcttatctgtaactacagttcagagagaactgacaccctcttctggcttcattcagcactgcatgctagtggtacacagacataatgcaggcagaacaccgatgcttgtaaaataaaaataaagatgaggtagttggggagattgctcaacagttaaaatcaatggttgctcctccgaaggatccaggtttgattcctagaacaaacatggtaactcaactagctatatttcaatcctaggggatccagtgccatctggggcctccatggacacttctcccttgtggtgaacaggcatagatacagccagaacattcatacatataaaataaaaataaaggtttttacacataaaataaaaataaagctctcgaagaggacctgagttcaattactaacactgcacccgaggtctcacaactccagctcgaaggggatctgaaactttctcattgcctcaggaggtaccagcacttgtgggcttgtactcacatacagataacagacatcattgagtacacctaattaagaagaagtcacttggaagtgtggcacacgccttaaatcccaatattcaggaacaaaaggcaggtgggtcttcaagttcaaggccaacctggtctacagcatgagttccagaacagccagggatacattaaaaatgaaggtgtcggggttggggatttagctcagcggtagagcgcttgcctagcaagtgcaaggccctgggttcggtccccagctccggaaaaaaaaaatgaaagtgtcttgttaaacaaaacaaaaagacaacaagcaaaaagattacttatgtgggcacgcactgggcttactttcttttctatttgagggacggttttattatgtgaccatggatgacctgagatttgctttgtagagtaagcttgccctgaacttttttttcccctggagctgaggacctaacccagggtggtgggtttataggcaagcgctctaccactgagctaaatccccaaccccccacccttcacttttaggataccaagcagactccttggtctaggaacaacctcagcctcgggactttttttttttttacactaggttccgctcctgttagactagactcttccacccctcagtacattatactactaggacactaggacaaaccatagcaaatctgtcacagcaccagtgacagccctaagcctgactccatcttttcttttctttttttaaatattatttattttatgtatatgagtacactgtcattgttctcagacacaccagaagagggcatcggatcccattacagatggttgtgagccaccatgtggttgctgggaattgaactcaggacctctgggagagcagtcagtgctcttaaccgctgagccatctctccagcccccactgaagacttttgatctggttaccatctgaccccaatctcttgcaaaagcctcccttcctccttcgaagaaactcttacgtcttttatgtccttggcccatgactttgtattaaatcagcaacaatgacaagacctgtatgtctctccctagctcagaagacagatccttgttccttgttaatgttttgattttctggtctgtccgtggggacagtctgatagttctaagactgatagctttgagggattctaaactcacaacagggctattgttaccgatgggcacaatacaaggctgccattgctttggagtgggaccattatcttgacagaaagaattaccataaaccctagctgtgattgctccgggagtccatgctaatgaaacactgcccacggccttcaggaaacttctcacagagtgctgcctcttggaatgactgtgtgaactctctactgtccacctgcagcagccataccgaaatacagtctaataacctctcaacttctgcattcttagtcttggtgaactctttcgcctccaatgtcatgacctttcaaagtcacctcacatagcagtctgcagcgagaacaggtaattcaggggctggggatttagctcagtggtagagcgcttacctaggaagcgcaaggccctgggttcggtccccagctccggaaaaaaaaaagaaccaaaaaaaaaaaaaaaagagagaacaggtaattcagctaagactggtgacacaagtgtaattttaatacttaggaggttgaggcgagcgcatctggagtttggattaacctggactccatagtgaatattgggctagcttaggctacataagcaagcctctctctctctctgtctgtgtctctgtctctatctctgtctctgtctctcaaccacaaaagagagaacggaaaaaaggaagaaattaagagaaagaaaaacaaaagaaatttctctaagcaaagcatatttatttatttatttattgtttttcaagacagtgtttgtctatgtagcattggctgtcctagaacaatcgttgtaggccaagctggccttgaactcataggcctgcctttgccttccaaatactggaattgaagccttgtggcagcactgcccagcgacacctggaattttttaaaatttatttatttatttatttatttatttatttatttatttatttatacactccagatattattcccctcttggtccatcccccaactgttccacatgtcataccttcccccaccccccagtctccacaaggatgtctccaacccacccaccctctctaatttttattgtacattcctctttctttctttttttttttttttttttttgggtctttttttccggagctggggaccgaacccagggccttgcgcttcctaggtaagcgctctaccactgagctaagtccccagcccctacattcctctttctaacttctttggcacagcatcttggagggtgcaaatcaagagacagcttttcttttcttttgtgatgccaactttcaagcatttacattttgggttgggttgggttgtgattttttttttgtcttcgaaatctgcattttttttctttcctttttttttttttttcagagctggggacctaacccagggccttgcgcttgctaggcaagcgctaaaacactgagctaaatccccaactcctaaatctgtatttttatttgtaacaactgtatttctttttctatatcctttaactctggagttttcatttcttccctcctgcccccataactatagtcacagttaaactgtgttatcaggaaattcaggaaaggtgccttgatgaacagatcaggacaggagctctgaccagtagtcactgtcttcctcttccttagaataagtaaaaatgaaaccaaccaaactttcttctctttctttctttcttttttttttttttttttttgacgtgtctcctgtgctttgtcagtagcatgaatttcattttttttttttttttttggtttaaaaaaggcaacctcaaaacccaaacctctttattgtcagggaaaagggaactgcaatgacttgaatttgaggatgtgggtactgcctcactcacacacattctcagactgtgtgatgccctgcacacctgtagaacagttacatgtatgtgcacctgtatttgtgcctattagaacaggacctgcagggaagtctacctaacccgaaactccccagtggaacaggcagggtgggtggagggctgggacagacaaggactcggcgcacacatacagtaccacataaaacagtacagtgaaggtgggctcaagacccaggcagcttccttcttttcagtaacagggcccaggctgcctttcacagcacaaccccacagctgaacccaggtctctcttcaaaaccagccatctcactcagcagcgccaaaggaaaagtagatgtagcctccctgcagagaaacagcttttcttgttgtttttaaataagtaagtaaatccaccatccctctgctccaagatggctgatgttacacttttctaccagattggtgcctgcttagctcactaacagtgctgcctccgccggctgtggcagagtttccagtgtggtgttttcaagcctcacccactcatcctctcattcccaaacattcagtgccctcctcacttaggggttttcgaaatgtttaaattttgtattactttaaatatatatttgttttattttcatgcgtctgtgtgtatgcttgtgagtttcacacatgctgtgtgtgcacaggaatctatgaaagccagaacagggcatcagatctacaggaagaaaccaagtgtccaaaaagggaagaaacgagatccatctgcctctgtggtgctggaattgaaggtgtacatcactacaaccaccggggatgggtatgtatgtatatatatatatatatatatatgtgtgtgtgtgtgtgtgtgtgtgtgtgtgtgtgtgtaagggtgtcagaccttctggaactggagttagacagttgtgagctgccatgtgggtgctgggaatgaaccctggccctctagaagaacagctgatgctcttaactgctgagccatctctccggccccttattttttatttgtgtgagagagtggaggtcaggggacaaactgagagacttggttctctccttctgccatgtgaatgccagggattgaatgcaggttgttagccttggcagtgagtgctttccccgcagggccatcttgtcagctctttgattacattgtaaaccctggcactgtgttatttgctgggaaatgtttttagttgtgggatgactcagctttagcacatgcctttaatccgagagctttctgcttgtatattgtaagcaggattaaataaagtcaaatcttaggtcaagagatggagcaagcaaagagttgacaggaaatgaacatagaattattgagaaaaaacatataggggttggggatttggctcagtggtagagcgcttacctaggaagcgcaaggtcctgggttcggtccccagcaccggaaaaaaaaaaaaaaaacatatagagtaagggggagtcgggtttaaactgtacagaagtctccatgtcttatttataatgtaagcaggtctgcaaaagcctgccgttgtgtcctgttgcctttcttctggcagtgaagaggatcagttttgaaggcaggcagaataggtgcggagagatggcttggcagttaagagtatatgctgctcttgcagaggacctgcatgcaactgccagcacccacacagtggttcgtagctacctgtaacttcgttccatgggatccgatgccttcttctgacctctgagagcaccgaccatgcacatagtgcatgaacatacatgcgggtgaaagactcacataaagtaaagtgaatacatctaattaaaataaagaccactttatgggctggagagatggctcagcggttaagagcactgactgctcttcctgaggttctgagttaaattcccagcaacagatggtggctcacaaccatctgtaatgagatgtgatcccctcttcctggtgtgtgtgaagacagctcccagtgtactcaatacacccctcccctccctgaatgggaaaaaaaaaaaaaaagcctggggttggggatttggctcagtggtaaaaaaaatacctatgaagcacaaggtcctgggttcggtccccagccccgaaaaaaaaaaagaaaaaaaaaaaagaccactttacacgtaaaaaataaaagatgggcagattaggccctgtactaaacaggattctttagaggaactgaaatgagtgtgtgtgtgtgtgtattcattttttttaaagatttatttattttatgtatatgaagacactgttgctatcttcagacacaccagaagagggcatcagatcgccttaaagatggctgtgagccatcatgtgggtactgggatttgaactcaggacctctggaaaagcagccccgtgtgtactcattttatatatgaaatatatacacacatacacacgtgtgtgttagattggcttccttgatggtccaggtaattcatcaatgagaatcagtagttactcagtctacaaagctgaatgtcgcgacaattctgatctggcactttagacctagaggactcctggagagtctacatgggaatcctggacatctggagatcctacacaaaatccctgccattcccaccaagggcagctgtgaatggctgtggggaaacattccttaagctaagcctgaagacctaaatccaatccctggaacccgtgtggtagatggagagaactgacttctgtttcatctgacctccactggtgtagccgcacatacatgcatgcaaaacagtcgtgataaataaatctaaaaaaagttagagcacctgtcaatagataagtataacttaaaagtgaaacgaagcctatgcttttaaatcgtaaggactgggaggcagtcaggcacatatccaggttccagaccagcctgatgtatgtaatgagttccagaccaattagggctatatcatgagaccatgtctcaaaaccaaaaaacaaaagaaaagaagaaaaaagaagaacatcaagtcaagcatgataaatcacataatcctataatcctaataatggggaggctgaagcagaatggccatgcctttgagcttagcctgggcaggacaaccaactgggctacacaggaatacataatacactgccattagaaaaaaaagcatggctgacttcgtcactgctagttggggcttgggtttaggtcttttcaaacactaagcaatttggttcggagctagtttttgagccctctgcccaccgccatggaggagccaccagagaaagtcgacccagttgtagtcccagaagctcctcaaatgaaagatgacgaggacgcgtccgctgattcagaagtcctacaaccagaaacactagtaaaggtcatgaaaacgctaaccctgaaccccagtgccgaacggtcagcacgtcatcacagcctcagtgtccggattcagggcaggcctgtggagaacagatgtgaaggaatcttgagggaagttcaaagcctttcccaagagaagggtccacacattgttggtggtgctgagagatgccggagcaaggatgagaagatttgttgggattgagcagagacaacaaaggcttgaaggaaatgagtaggaagggaagagtgagccactcagacgtctctgtgcttcctgccatcgtcagagatggaatccgtctaagaaagctaaaatccggaagaattaggacagtcggtttatgtacactatccttgctgctcatgatgccatgcagcagacctgaaaactggtttttgttttttaaagataaaacttttcctggtgctggggaacacgtcttgttaacctttcaactatgtaggaagtgtgacggttgaattcatgtgaaggacttaaatttacccaaagtatggagaatgagttaaagcattctgtgaactttagaagcctcaagctgggggctgagaaacactgtaactagaatttggggtagtttgctttagaaggtaattggaataggcctttggattttctagtttgcagaaatgtgtaataaaggcaattttgttatctttaacaaacacacagaacagattagaatgagccattggagatggggggttgtttttacaggagcacgtgtgggtgcgcacactcctgatgtccagagttcaatgtgtgttgctaaccctgtttatttctgctccaggcagggtctccatgagcctagccagtctctcagctcgtggtcctgcctcccttgttgcccaagttttgacgccacaggcttgacagcaagatctagaaaatgcttgtcttgattttgtgtttgttcatgctgtgtaataaaaagaacaattggttgatgtattcctaaatttaaaaaaaaaaaaaaaagcaccaggtgatggtggctcacccctttaatcccaacgctcagaaggcagagacgggtggatctctgaattcatggccagccagggctacacagcaaaaccctgtcttgagaaaagagacttgtggggttggggatttggctcagtggtagagcgcttgctaccctgggttcggtccccagctccgaaaaaaagaatagaaaaaaaaagaaaaaagaaaaaagagactcgtaagcaagcaaagcttggtagtctaaagaaatgagaaatccttagagctaccttagagctagaaaaggcaggacatttcaggcagagagctggtacggcaagcccaaaggctcagggcccggtttataccatgtaaggttatcctgaggggctggagaagaaatgcacagcaacactaacacgtcatactgtctggccaagtatcaactaccatggctttatagatcctgctcttgaggaaaggggtagatcaaggggtaatcaaggatagattacccctttggcaataggacggagggtggctagatccctccaacagtgtgagtaggtccaagagtatgaatcatctatggctcctaataaacactgctaggctaatttaccattgagctacatcccaaatatcaaaagttgttttgggagaggggatgcatgggagacaggttctaatgtgaatcttactgtcctggaactccctccatagaccgtgctggctttgaacttacagagttctcacaggagacttaactgcctttgtctccaaagtgctgggatcaaaggcgtgcaccaccacatccagccttattttaattaattataatcaattattaattaattataatcataattttaattagttttgatcatatttatcgatgtattatggaagtggggccttgcatgtcattcttgttggtaaaggtcaggagataaaaatactacttggtaaataagaaaacccaagttaagaaagatggagaaaaaaaaacaatattatagttaaaaaaaaaaaaacttggtcttttaaaaataaaatacagggggctggggatttagctcagtggtagagcgcttacctaggaagcacaaggccctgggttcggtccccagctctgaaaaaaagaaccaaaaaaaaaaaaagaaaaaagaaaatacagggctggagagatgctcagcggctaagagcactgactgctcttccagaggtcctgagttcaattcccagcaaccacatggtggctcacaaccatttgtaatgggatctgatgccctcttctggtgtgtctgaagacagctacagtgtacatgaatacataaataaattctttaaaaaaatgaaaaataaaatacatgtcatatgatttatcaaaaaaaaaatactacttggacagggttggagatttagctcagtggccgagcacttgcctagcaagtgcaagaccctgggttcggtcctcagctctgaaaaaaaaaattactacttggagaagtaggttctccccttccactcaagttgtagaaatccaacttagatgtcaggaggcaagctctcgtaccaacggaacttaagattttggtttttgaagtcttgtagagaccaggctatcctgaaatcaagatttaatttacccagctccaaaaaaaaaaaaaaaagatttaatttaaagtagctgttccatgcctttgatcccagcactctggacaagagaggcagatgcaggttggtgtgtgagtttgagatcagtctcaaagcttggtccacatggaaagttctagaacagccaaggcttcatgagatcgtgtctcaaaacagcaaagacagtgacgatgacgtgatgatgatgagcaacatagactcaagcgtgctaggccaaaacaccactagatctgctccctagcccctgacaagtaatttgctaacaacatgcatagtggttattcttccaatttctccttctccttctccttctcctccttctccttcttcttctgtttatttatttatgtgagtacactgtagctgtcctcagacacaccagaagagggcatcggatctcattacagatggctgtgagccaccatgtggttgctgggatttgaactcaggacctctggaagagcagtcagtgctcttagctgctgagcgtctctccagcccccaatttcttcttttaaaattacataatcaccactaggtggggtggcacatgcaggcagatctctgtgggtttgaggtctgcctggtcttggtattgagttccaggtcagccagagctatattctgagaccctgtctcaaaaagacagaaatagaagtaaaaaagaaaacggaaaattaaaaaacacagggaggcggtggtgacacactttgatcccagtactgcatttgggaggcagaggcaggtggatctctttgtattacaggccagcctggtctacagagaattccaggacatcaagtactatgcagagaaactctgtctcaaaacaccaataaacaaacaaacaaacaaacaagtaaaaataaataaataaaaattaaaaaaggaaaagaaaaacgaaaaagaaagaagagaataaaattgtattgcttatcatgaatgctccaactcgtgtgtttaggtcagaagacaactaacaggaatccttttttctctggtatcaaactcgtgggtcttaggaatcgaactcacatacttcggttgggcggcaagcgattttacccgctgatccatgacacaggccctctttaatttctaaagccctacatgcgggtctggactttattcacggtgggtgggtcttcttcctgtcagtttccgtccgcagatgtccccgcccaccaggaaggatctttcgggctctcgtcggcacccgtccaccctgtctccacgtgacacaaacagacagggcacttccgcttcccgtccactctcctcactcagtgtctacaccccccgtccccgggtcccccgcccggtgagttagcgagcgccgggagggcggcgtcgcgggcggagtcgccccgggctgacccttgccgccttccttcttctcaccgcaggtccccgcggtagcggaggcgggcgccatggcggagctgacggctctggagagcctcatcgagatgggctttcccaggggacgcgcgtaagggaacctcccctctagcctgtggtgggaggccgcgggcctgccgggcctcactgtcaccatggctggtgggcgctattcacggtgtttctgccctcagggagaaggctctggccctcacagggaaccagggcatcgaggctgcgatggactggtgagcgactggcacgggtggaggaagtttgggggcctctgggaaaggcggcctcaaggctaaccccctgccaactttctctgcccaggcttatggagcatgaagacgaccccgatgtggacgagcctctagagactcctctcagccatatcctgggacgagaacccacgccctcagagcaagttggtcctgaaggtcctgactgggagacatcttgtgattctagctatctagtgagggcctgaggaaaccagaatgctttcactataaataataatactagttgcttgtttgtaggatctgggtctgctgctggagaaagcaaacccgttttgactgaagaggagaggcaagaacagactaagaggtaactgtgcaagttcagtgtgtgtgtgtgtgtgtgtgtgtgtgtgtgtgtgtgtgtgtgtgtgtgtgtgtgtgtgtgttttgtttggagcctgcctcactcctgtccaggctgaactctggatcctgctgcctcagcctccagagtgctgggattacaggtcttcaccactgtgccctgtattattttttgagacagggtctagctgtgtacctcaggctggcctggaacctaggctaaatgcaacgccacattcttctgagtgttgtgatcaccatagctagcccattaacacactttcccaagggtcatgggtcatcttcctttcttctcaaatacaaacacaagtcaggacagacctggcctttccagttagtggatgttgggggagtcaccaggaaacatctcatacagcacaagactgtctaaactcctgcgtggctgcagactcccctgaaatcccaattctctggcccctactttgcaagtgcagggactgtaggtattcaccaccgtgcctggctcttgtctgccctttttaaaaaaacaaaaaacaaaaaggccccatgcataatgtatgtgctctaacactgagctacctttttttttttcttttggttttggttttgtttttttcaagccagagtctgtctctatccccgctgtccttaaactggctctatagacctggctggactgaaactcaagaaatccacctgcctctgccttctgagcactgaggggtgcactgtcaccacctagcttgccctttttatgttactgtcttggctttgtttttttttttcttttttttttcttttttttggagctggggaccgaacccagggccttgtgcttgctaggtaagcgctctaccatcgagctaaacccccaaccgggctttgttttcttttatctgtcttggaacacaatcctttaatctgttaattctctgtttaaactcaccttcccactccatatccagcttcagctttttcttctctgcaaaacagaatgttggaacttgtggcgcagaagcagcgggaacgtgaagaaagagaggagcgagaagctttagaacgagagaagcagcggaggagacaagggcaagagctgtcagctgcacgacagaaactacaggaagatgagatacgccgggctgctgaggagcgcaggagggagaaggctgaagagctagctgccaggtctgaagactcataggtcactaacggaggaagaaatgaagacttgccttgcccatgtctgacctatcttcctcctgtctctcttctagacaaagggggcgagagaaaattgaaagggacaaagcagagagagcccagaaggtgggtgatgaggaagtctgtgggtataatggagtaggggggtgcggggccgtgggggcgtgcgggcgaggggggggggggggggcgcgggtgggcgggggacggagagggggcggggcaggcggggggggggcgcggaggtgcggggggtttctcacgggtggaggaggggcggggggggggggaggtggggtcgtgcggttgatggtgcggcggggttgatagacgccgtgcgagttggcggcggggggcgggcggtggaggggcggctgagacggggggcagggggtgcgttgggggtggagggcagtggggcgggtgcggttgctggcgcgggcggcgcggaacggtagccggggcgcgcgggagcgcgcgcgcgcgctcgcgagggggtgcggccggagaggggtgcggaggtccggtgagctgactgacgatgcccggtagctgctggcgcgtgggcgacgcgtcatgccgtggcgcgggtggggcgggcgcggtgcatgcgcgagcgtcctcggtctggcgaccgtagcgcgctctctgtcggggccgcggaccggcggtgagggtcgggggcgggggtgcgtggtggctggaaggcgagtggtgtcgggtagagggcggcgatagggggcgcgcgtgatgtgatat

SEQ ID NO: 10 - protein, 17 aa, Mus musculus
ASGGQPPNYERIKEEYE

SEQ ID NO: 11 - protein, 16 aa, Mus musculus
RDRKMVGDVTGAQAYA

SEQ ID NO: 12 - protein, 16 aa, Mus musculus
MEEPSEKVDPMKDPET

SEQ ID NO: 13 - protein, 17 aa, Mus musculus
CHYQRWDPSENAKIGKN

SEQ ID NO: 14 - DNA, 60 bp, Artificial Sequence
  AL1 PCR Primer
attggatccaggccgctctggacaaaatatgaatcctttttttttttttttttttttttt

SEQ ID NO: 15 - DNA, 22 bp, Artificial Sequence
  BMP4 5′ Primer
gccataccttgacccgcagaag

SEQ ID NO: 16 - DNA, 22 bp, Artificial Sequence
  BMP4 3′ Primer
aaatggcactcagttcagtggg

SEQ ID NO: 17 - DNA, 24 bp, Artificial Sequence
  TNAP 5′ Pimer
cccaaagcaccttatttttctacc

SEQ ID NO: 18 - DNA, 21 bp, Artificial Sequence
  TNAP 3′ Primer
ttggcgagtctctgcaattgg

SEQ ID NO: 19 - DNA, 21 bp, Artificial Sequence
  Oct4 5′ Primer
cactctactcagtcccttttc

SEQ ID NO: 20 - DNA, 22 bp, Artificial Sequence
  Oct4 3′ Primer
tgtgtcccagtctttatttaag

SEQ ID NO: 21 - DNA, 21 bp, Artificial Sequence
  Hoxb1 5′ Primer
aactcatcagaggtcgaagga

SEQ ID NO: 22 - DNA, 21 bp, Artificial Sequence
  Hoxb1 3′ Primer
cggtgctattgtaaggtctgc

SEQ ID NO: 23 - DNA, 19 bp, Artificial Sequence
  GCR1 5′ Primer
ctactccgtgaagtctagg

SEQ ID NO: 24 - DNA, 21 bp, Artificial Sequence
  GCR1 3′ Primer
aatgagtgttacacctgcgtg

SEQ ID NO: 25 - DNA, 21 bp, Artificial Sequence
  GCR2 5′ Primer
gccattcagatgtctctgcac

SEQ ID NO: 26 - DNA, 21 bp, Artificial Sequence
  GCR2 3′ Primer
ctcacagcttgaggcttctaa

(19) United States
(12) Patent Application Publication
(10) Pub. No.: US 2005/0054823 A1
(43) Pub. Date: Mar. 10, 2005

(54) Genes

(76) Inventors: Mitinori Saitou, Cambridge (GB); Azim Surani, Cambridge (GB)

Correspondence Address: FROMMER LAWRENCE & HAUG, 745 FIFTH AVENUE- 10TH FL., NEW YORK, NY, 10151, US

(21) Appl. No.: 10/621,911

(22) Filed: Jul. 17, 2003

Related U.S. Application Data
(63) Continuation-in-part of application No. PCT/GB02/00215, filed on Jan. 18, 2002.

(30) Foreign Application Priority Data
Jan. 18, 2001 (GB) 0101300.2

(51) Int. Cl. C07K 14/705; C07H 21/04

(52) U.S. Cl. 530/350; 435/69.1; 435/320.1; 435/325; 536/23.5

(57) ABSTRACT

The present invention describes and claims two primordial germ cell-specifically expressed genes, GCR1 (Fragilis) and GCR2 (Stella), which are markers for primordial germ cells and may be used to identify such cells in cell populations.

### REFERENCE TO RELATED APPLICATIONS

This application is a continuation-in-part application of International Patent Application PCT/GB02/000215, filed Jan. 18, 2002 which published as WO 02/057307 on Jul. 25, 2002, and which claims priority to Great Britain Patent Application 0101300.2 filed Jan. 18, 2001, both of which are incorporated herein by reference.

For the purposes of this application, it is noted that sequence homology or identity such as nucleotide sequence homology also can be determined using the “Align” program of Myers and Miller, (“Optimal Alignments in Linear Space ”, CABIOS 4, 11-17, 1988, incorporated herein by reference) and available at NCBI, as well as the same or other programs available via the Internet at sites thereon such as the NCBI site.

Alternatively or additionally, the term “homology ” or “identity”, for instance, with respect to a nucleotide or amino acid sequence, can indicate a quantitative measure of homology between two sequences. The percent sequence homology can be calculated as (N_{ref}−N_{dif})*100N_{ref}, wherein N_{dif} is the total number of non-identical residues in the two sequences when aligned and wherein N_{ref} is the number of residues in one of the sequences. Hence, the DNA sequence AGTCAGTC will have a sequence identity of 75% with the sequence AATCAATC (N_{ref}=8; N_{dif}=2).

### FIELD

The present invention relates to the fields of development, molecular biology and genetics. More particularly, the invention relates to genes which are expressed exclusively in the earliest populations of primordial germ cells (PGCs) and the use of such genes and the products thereof in identification of pluripotent and multipotent cells such as PGCs, pluripotent embryonic stem cells (ES) and pluripotent embryonic germ cells (EG), in cell populations. They are also markers for a change in the sate of cells from being non pluripotent to becoming pluripotent, and in being able to confer this state on a non pluripotent cell.

### INTRODUCTION

Post fertilisation, the early mammalian embryo undergoes four rounds of cleavage to form a morula of 16 cells. These cells, following further rounds of division, develop into a blastocyst in which the cells can be divided into two distinct regions; the inner cell mass, which will form the embryo, and the trophectoderm, which will form extra-embryonic tissue, such as the placenta.

The cells that form part of the embryo up until the formation of the blastocyst are totipotent; in other words, each of the cells has the ability to give rise to a complete individual embryo, and to all the extra-embryonic tissues required for its development. After blastocyst formation, the cells of the inner cell mass are no longer totipotent, but are pluripotent, in that they can give rise to a range of different tissues. A known marker for such cells is the expression of the enzyme alkaline phosphatase and Oct4.

Primordial germ cells (PGCs) are pluripotent cells that have the ability to differentiate into all three primary germ layers. In mammals, the PGCs migrate from the base of the allantois, through the hindgut epithelium and dorsal mesentery, to colonise the gonadal anlague. The PGC-derived cells have a characteristically low cytoplasm/nucleus ratio, usually with prominent nucleoli. PGCs may be isolated from the embryos by removing the genital ridge of the embryo, dissociating the PGCs from the gonadal anlague, and collecting the PGCs. The earliest PGC population is reported to consist of a cluster of some 45 (forty-five) alkaline phosphatase positive cells, found at the base of the emerging allantois, 7.25 days post-fertilisation (Ginsburg et al., (1990) Development 110:521-528).

PGCs have many applications in modern biotechnology and molecular biology. They are useful in the production of transgenic animals, where embryonic germ (EG) cells derived from PGCs may be used in much the same manner as embryonic stem (ES) cells (Labosky et al., (1994) Development 120:3197-3204). Moreover, they are useful in the study of foetal development and the provision of pluripotent stem cells for tissue regeneration in the therapy of degenerative diseases and repopulation of damaged tissue following trauma. Above all, PGCs while having some specialised properties, retain an underlying pluripotency, which is lost from the neighbouring cells that surround the founder population of PGCs that acquire a somatic cell fate. PGCs and the surrounding somatic cells share a common ancestry. However, the founder PGCs are few in number and difficult to isolate from embryonic tissue and the surrounding somatic cells, which complicates their study and the development of techniques which make use thereof.

Little is known in the art about the expression of genes in the founder population of PGCs and the relationship between PGC-specific gene expression and the retention of pluripotency in these cells. Certain markers for PGCs are known—for example, the expression of tissue non-specific alkaline phosphatase (TNAP) has been used as a marker for early PGCs (Ginsburg et al., (1990) Development 110:521-528). Oct4 is known to be expressed in PGCs, but not somatic cells (Yoem et al., (1996) Development 122:881-894). Other markers, such as BMP4, are known to be expressed primarily in somatic tissues (Lawson et al., (1999) Genes & Dev. 13:424-436). However, none of these genes is specific for PGCs, since they are also expressed in other tissue types. There is therefore a need in the art for the identification of genes which may be used as markers for PGCs and which may provide an insight into the biology of germ cell development and the nature of the pluripotent state.

### SUMMARY

We disclose the sequences of two genes which are expressed specifically in PGCs and other pluripotent cells. The sequence of the genes from mouse is set forth in SEQ ID NO: 1 (GCR1 or Fragilis) and SEQ ID NO: 3 (GCR2, or Stella). Corresponding amino acid sequences for mouse GCR1 and GCR2 are set out in SEQ ID NO: 2 and SEQ ID NO: 4 respectively. Nucleic acid sequences of rat GCR2 homologues are set out in SEQ ID NO: 5, SEQ ID NO: 6, SEQ ID NO: 7, SEQ ID NO: 8, and SEQ ID NO: 9.

According to a first aspect of the present invention, we provide a GCR1 polypeptide, or a fragment, homologue, variant or derivative thereof. Preferably, the polypeptide has at least 50%, 60%, 70%, 80%, 90% or 95% homology to a sequence shown in SEQ ID NO: 2.

There is provided, according to a second aspect of the present invention, GCR2 polypeptide, or a fragment, homologue, variant or derivative thereof. Preferably, the polypeptide has at least 50%, 60%, 70%, 80%, 90% or 95% homology to a sequence shown in SEQ ID NO: 4.

We provide, according to a third aspect of the present invention, a nucleic acid encoding a polypeptide according to any preceding claim.

As a fourth aspect of the present invention, there is provided a nucleic acid having at least 90% homology with the sequence set forth in SEQ ID NO: 1, or a fragment, variant or derivative thereof.

We provide, according to a fifth aspect of the present invention, a nucleic acid having at least 75% homology with the sequence set forth in SEQ ID NO: 3, SEQ ID NO: 5, SEQ ID NO: 6, SEQ ID NO: 7, SEQ ID NO: 8 or SEQ ID NO: 9, or a fragment, variant or derivative thereof

The present invention, in a sixth aspect, provides a nucleic acid comprising a sequence of 25 contiguous nucleotides of a nucleic acid according to the third, fourth or fifth aspect of the invention.

In a seventh aspect of the present invention, there is provided a nucleic acid comprising a sequence of 15 contiguous nucleotides of a nucleic acid according to the third, fourth, fifth or sixth aspect of the invention.

According to an eighth aspect of the present invention, we provide a complement of a nucleic acid sequence according to any of the third to seventh aspect of the invention.

Preferably, such a nucleic acid comprises one or more nucleotide substitutions, wherein such substitutions do not alter the coding specificity of said nucleic acid as a result of the degeneracy of the genetic code.

We provide, according to a ninth aspect of the invention, a polypeptide encoded by a nucleic acid according to any preceding aspect of the invention.

Preferably, the polypeptide comprises a sequence shown in SEQ ID NO: 2 or SEQ ID NO: 4.

There is provided, in accordance with a tenth aspect of the present invention, a method for identifying a pluripotent cell, comprising detecting the presence of a polypeptide according to the first, second, ninth or tenth aspect of the invention or the expression of a nucleic acid according to any of the third to eighth aspect of the invention, or a homologue thereof.

Preferably, the method comprises the steps of amplifying nucleic acids from a putative pluripotent cell using 5′ and 3′ primers specific for GCR1 (Fragilis) and/or GCR2 (Stella), and detecting amplified nucleic acid thus produced. Preferably, the expression of the nucleic acid sequence is detected by in situ hybridisation.

The expression of the nucleic acid sequence may be determined by detecting the protein product encoded thereby. Alternatively or in addition, the protein product may be detected by immunostaining.

As an eleventh aspect of the invention, we provide an antibody specific for a polypeptide according to the first, second, ninth or tenth aspect of the invention. preferably, the antibody is capable of specifically binding to an extracellular domain of GCR1.

We provide, according to a twelfth aspect of the invention, there is provided use of such an antibody for the identification and/or isolation of a pluripotent cell.

We further provide, according to a thirteenth aspect of the invention, a pluripotent cell identified by a method as set out previously.

There is provided, according to a fourteenth aspect of the present invention, a method for isolating a gene specifically expressed in a pluripotent cell, comprising the steps of: (a) providing a population of cells containing a pluripotent cell; (b) isolating one or more pluripotent cells therefrom and providing single-cell pluripotent cell isolates; (c) amplifying the transcribed nucleic acid present in a single pluripotent cell; (d) conducting a subtractive hybridisation screen to identify transcripts present in pluripotent cells but not in somatic cells; and (e) probing a nucleic acid library with one or more transcripts identified in (d) to clone one or more genes which are specifically expressed in pluripotent cells.

In a highly preferred embodiment, the pluripotent cell is selected from the group consisting of: a primordial germ cell (PGC), an embryonic stem cell (ES) and an embryonic germ cell (EG). Preferably, the pluripotent cell comprises a primordial germ cell.

### BRIEF DESCRIPTION OF THE FIGURES

FIG. 1: Nucleotide and deduced amino acid sequence of Fragilis. Predicted positions of the two transmembrane domains (TM I and TM II) are underlined and indicated by bold letters. The poly(A) signal is underlined.

FIG. 2: Nucleotide and deduced amino acid sequence of Stella. Three nuclear localization signals are underlined. A potential nuclear export signal is underlined twice, and the hydrophobic residues are indicated in bold. Helical structures in a motif with similarity to SAP domain (a.a.28 to a.a.63) are underlined in red, and the conserved residues are indicated by blue. A splicing factor-like motif is underlined and the conserved residues are indicated in green. Poly(A) signals are also underlined.

FIG. 3: Expression of Fragilis in embryonic stem (ES) cells. ES cells are fixed in 4% paraformaldehyde in PBS for 10 min. at room temperature and processed for immunohistochemistry as described by Saitou et al., (1998). J Cell Biol 141, 397-408. (1998). Fragilis expression is similarly detected in E6.5 proximal epiblast cells, which are germ cell competent cells, and in newly specified germ cells. The expression declines after E8.5 following completion of the specification of germ cells fate.

FIG. 4: Expression of Stella in PGCs. PGCs from E12.5 genital ridges are fixed in 4% paraformaldehyde in PBS for 10 min. at room temperature and processed for immunohistochemistry as described by Saitou et al., (1998). J Cell Biol 141, 397-408. (1998). Stella is detected in PGCs from E 7.25-13.5, as well as in pluripotent ES cells and in EG cells. Stella is also detected in the totipotent oocyte, zygote and in the totipotent and pluripotent blastomeres during preimplantation development and in developing gametes. When EG cells are derived from PGCs (Labosky et al., (1994) Development 120:3197-3204). Fragilis expression is again detected in the pluripotent EG cells as it is in ES cells. Therefore, Fragilis and Stella are also markers for the pluripotent stem cells.

FIG. 5. Fragilis expression by whole-mount in situ hybridization in E7.2 mouse embryos.

FIG. 6. Stella expression by whole mount in situ hybridisation in E 7.2 mouse embryos.

FIG. 7. Stella expression in PGCs in the process of migration into the gonads in E9.0 embryos.

FIGS. 8a and 8b. Expression of Fragilis and Stella in single cells detected by PCR analysis of single cell cDNAs. Numbers marked by symbol* in 8b are the PGCs. Note that there are more single cells showing expression of Fragilis compared to those showing expression of Stella. Only cells with the highest levels of Fragilis expression were found to express Stella and acquire the germ cell fate. Cells that express Stella were found not to show expression of Hoxb1. Cells that express lower levels of Fragilis and no Stella become somatic cells and showed expression of Hoxb1. The founder population of PGCs also show high levels of Tnap. Both the founder PGCs and the somatic cells show expression of Oct4, T(Brachyury), and Fgf8.

### DETAILED DESCRIPTION

GCR1 (Fragilis) AND GCR2 (Stella)

The disclosure provides generally for GCR1 (Fragilis) and GCR2 (Stella) nucleic acids, polypeptides, as well as fragments, homologues, variants and derivatives thereof.

The names “GCR1” and “Fragilis” should be understood as synonymous with each other, and likewise, “GCR2” and “Stella” should be considered synonyms. Nucleic acid and amino acid sequences of GCR1/Fragilis are set out in SEQ ID NO: 1 and 2, while nucleic acid sequences of GCR2/Stella are set out in SEQ ID NO: 3, 5, 6, 7, 8 and 9, with an amino acid sequence of GCR2/Stella shown in SEQ ID NO: 4.

In preferred embodiments, however, GCR1/Fragilis should be taken to refer to the nucleic acid sequence shown in SEQ ID NO: 1, or the amino acid sequence shown in SEQ ID NO: 2, as the context requires. Furthermore, in preferred embodiments, GCR2/Stella should be taken to refer to the nucleic acid sequence shown in SEQ ID NO: 3, or the amino acid sequence shown in SEQ ID NO: 4, as the context requires.

GCR1 and GCR2 are PGC-specific transcripts. GCR1 is upregulated during the process of lineage commitment of PGCs, while GCR2 is upregulated after GCR1, and marks commitment to the PGC fate. The first gene, GCR1 (Germ cell restricted-1, Fragilis), encodes a 137 amino acid protein with a predicted molecular weight of 15.0 kD. The best fit model of the EMBL program PredictProtein predicts two transmembrane domains, both N and C terminus ends being located outside. The BLASTP search revealed that Fragilis is a novel member of the interferon-inducible protein family. One prototype member, human 9-27 (identical to Leu-13 antigen), is inducible by interferon in leukocytes and endothelial cells, and is located at the cell surface as a component of a multimeric complex involved in the transduction of antiproliferative and homotypic adhesion signals (Deblandre, 1995). The BLASTN search revealed that the Fragilis sequence was found in ESTs derived from many different tissues both from embryos and adults, indicating that Fragilis may play a common role in different developmental and cell biological contexts. Database searches reveal a sequence match with the rat interferon-inducible protein (sp:INIB RAT, pir:JC1241) with unknown function. The GCR1 sequence appears six times in our screen, indicating high level expression in PGCs.

The second gene, GCR2, (Stella) encodes a 150 amino acid protein, of 18 kD. It has no sequence homology with any known protein, contains several nuclear localisation consensus sequences and is highly basic pI (pI=9.67, the content of basic residues=23.3%), indicating a possible affinity to DNA. Furthermore a potential nuclear export signal was identified, indicating that Stella may shuttle between the nucleus and the cytoplasm. BLASTN analysis revealed that the Stella sequence was found only in the preimplantation embryo and germ line (newborn ovary, female 12.5 mesonephros and gonad etc.) ESTs indicating its predominant expression in totipotent and pluripotent cells. Interestingly, we found that Stella contains in its N terminus a modular domain which has some sequence similarity with the SAP motif. This motif is a putative DNA-binding domain involved in chromosomal orgainisation. Furthermore, the SMART program revealed the presence of a splicing factor motif-like structure in its C-terminus, These findings indicate a possible involvement of Stella in chromosomal organisation and RNA processing.

Antibodies may be raised against the GCR1 and/or GCR2 polypeptides. In particular, antibodies may be raised against the extracellular domain of GCR1, which is a transmembrane polypeptide.

Antibodies and nucleic acids disclosed here are useful for the identification of PGCs in cell populations. The methods and compositions described here therefore provide a means to isolate PGCs, useful for example for the study of germ tissue development and the generation of transgenic animals, and PGCs when isolated by a method described here.

Homologues of GCR1 and GCR2 may also be used to identify PGCs and other pluripotent cells, such as ES or EG cells.

The practice of the present invention will employ, unless otherwise indicated, conventional techniques of chemistry, molecular biology, microbiology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; J. M. Polak and James O'D. McGee, 1990, In Situ Hybridization: Principles and Practice; Oxford University Press; M. J. Gait (Editor), 1984, Oligonucleotide Synthesis: A Practical Approach, Irl Press; and, D. M. J. Lilley and J. E. Dahlberg, 1992, Methods of Enzymology: DNA Structure Part A: Synthesis and Physical Analysis of DNA Methods in Enzymology, Academic Press. Each of these general texts is herein incorporated by reference.

Polypeptides

It will be understood that polypeptide sequences disclosed here are not limited to the particular sequences set forth in SEQ ID NO: 2 and SEQ ID NO: 4, or fragments thereof, or sequences obtained from GCR1 or GCR2 protein, but also include homologous sequences obtained from any source, for example related cellular homologues, homologues from other species and variants or derivatives thereof.

This disclosure therefore encompasses variants, homologues or derivatives of the amino acid sequences set forth in SEQ ID NO: 2 and SEQ ID NO: 4, as well as variants, homologues or derivatives of the amino acid sequences encoded by the nucleotide sequences disclosed here.

Homologues

The polypeptides disclosed include homologous sequences obtained from any source, for example related viral/bacterial proteins, cellular homologues and synthetic peptides, as well as variants or derivatives thereof. Thus polypeptides also include those encoding homologues of GCR1 and/or GCR2 from other species including animals such as mammals (e.g. mice, rats or rabbits), especially primates, more especially humans. More specifically, homologues include human homologues.

In the context of the present document, a homologous sequence or homologue is taken to include an amino acid sequence which is at least 60, 70, 80 or 90% identical, preferably at least 95 or 98% identical at the amino acid level over at least 30, preferably 50, 70, 90 or 100 amino acids with GCR1 or GCR2, for example as shown in the sequence listing herein. In the context of this document, a homologous sequence is taken to include an amino acid sequence which is at least 15, 20, 25, 30, 40, 50, 60, 70, 80 or 90% identical, preferably at least 95 or 98% identical at the amino acid level, preferably over at least 50 or 100, preferably 200, 300, 400 or 500 amino acids with the sequence of GCR1 or GCR2, for example GCR1 (SEQ ID NO: 2) and GCR2 (SEQ ID NO: 4). Although homology can also be considered in terms of similarity (i.e. amino acid residues having similar chemical properties/functions), in the context of the present document it is preferred to express homology in terms of sequence identity.

Homology comparisons can be conducted by eye, or more usually, with the aid of readily available sequence comparison programs. These commercially available computer programs can calculate % homology between two or more sequences.

% homology may be calculated over contiguous sequences, i.e. one sequence is aligned with the other sequence and each amino acid in one sequence directly compared with the corresponding amino acid in the other sequence, one residue at a time. This is called an “ungapped” alignment. Typically, such ungapped alignments are performed only over a relatively short number of residues (for example less than 50 contiguous amino acids).

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package (see below) the default gap penalty for amino acid sequences is −12 for a gap and −4 for each extension.

Calculation of maximum % homology therefore firstly requires the production of an optimal alignment, taking into consideration gap penalties. A suitable computer program for carrying out such an alignment is the GCG Wisconsin Bestfit package (University of Wisconsin, U.S.A.; Devereux et al., 1984, Nucleic Acids Research 12:387). Examples of other software than can perform sequence comparisons include, but are not limited to, the BLAST package (see Ausubel et al., 1999 ibid—Chapter 18), FASTA (Atschul et al., 1990, J. Mol. Biol., 403-410) and the GENEWORKS suite of comparison tools. Both BLAST and FASTA are available for offline and online searching (see Ausubel et al., 1999 ibid, pages 7-58 to 7-60). However it is preferred to use the GCG Bestfit program.

Although the final % homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see user manual for further details). It is preferred to use the public default values for the GCG package, or in the case of other software, the default matrix, such as BLOSUM62.

Once the software has produced an optimal alignment, it is possible to calculate % homology, preferably % sequence identity. The software typically does this as part of the sequence comparison and generates a numerical result.

Variants and Derivatives

The terms “variant” or “derivative” in relation to the amino acid sequences as described here includes any substitution of, variation of, modification of, replacement of, deletion of or addition of one (or more) amino acids from or to the sequence. Preferably, the resultant amino acid sequence retains substantially the same activity as the unmodified sequence, preferably having at least the same activity as the GCR1 and/or GCR2 polypeptides shown in the sequence listings. Thus, the key feature of the sequences—namely that they are specific for PGCs and other pluripotent cells, such as ES or EG cells, and can serve as a marker for these cells in a cell population—is preferably retained.

Polypeptides having the amino acid sequence shown in the Examples, or fragments or homologues thereof may be modified for use in the methods and compositions described here. Typically, modifications are made that maintain the biological activity of the sequence. Amino acid substitutions may be made, for example from 1, 2 or 3 to 10, 20 or 30 substitutions provided that the modified sequence retains the biological activity of the unmodified sequence. Amino acid substitutions may include the use of non-naturally occurring analogues, for example to increase blood plasma half-life of a therapeutically administered polypeptide.

Natural variants of GCR1 and GCR2 are likely to comprise conservative amino acid substitutions. Conservative substitutions may be defined, for example according to the Table below. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

Fragments

Polypeptides disclosed here and useful as markers also include fragments of the above mentioned full length polypeptides and variants thereof, including fragments of the sequences set out in SEQ ID NO:2 and SEQ ID NO: 4.

Polypeptides also include fragments of the fill length sequence of any of the GCR1 and/or GCR2 polypeptides. Preferably fragments comprise at least one epitope. Methods of identifying epitopes are well known in the art. Fragments will typically, comprise at least 6 amino acids, more preferably at least 10, 20, 30, 50 or 100 amino acids.

Included are fragments comprising, preferably consisting of, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145 or 150, or more residues from a GCR1 and/or GCR2 amino acid sequence.

Polypeptide fragments of the GCR proteins and allelic and species variants thereof may contain one or more (e.g. 5, 10, 15, or 20) substitutions, deletions or insertions, including conserved substitutions. Where substitutions, deletion and/or insertions occur, for example in different species, preferably less than 50%, 40% or 20% of the amino acid residues depicted in the sequence listings are altered.

GCR1 and/GCR2, and their fragments, homologues, variants and derivatives, may be made by recombinant means. Howeve,r they may also be made by synthetic means using techniques well known to skilled persons such as solid phase synthesis. The proteins may also be produced as fusion proteins, for example to aid in extraction and purification. Examples of fusion protein partners include glutathione-S-transferase (GST), 6×His, GAL4 (DNA binding and/or transcriptional activation domains) and β-galactosidase. It may also be convenient to include a proteolytic cleavage site between the fusion protein partner and the protein sequence of interest to allow removal of fusion protein sequences. Preferably the fusion protein will not hinder the function of the protein of interest sequence. Proteins may also be obtained by purification of cell extracts from animal cells.

The GCR1 and/or GCR2 polypeptides, variants, homologues, fragments and derivatives disclosed here may be in a substantially isolated form. It will be understood that such polypeptides may be mixed with carriers or diluents which will not interfere with the intended purpose of the protein and still be regarded as substantially isolated. A GCR1/GCR2 variant, homologue, fragment or derivative may also be in a substantially purified form, in which case it will generally comprise the protein in a preparation in which more than 90%, e.g. 95%, 98% or 99% of the protein in the preparation is a protein.

The GCR1/GCR2 polypeptides, variants, homologues, fragments and derivatives disclosed here may be labelled with a revealing label. The revealing label may be any suitable label which allows the polypeptide , etc to be detected. Suitable labels include radioisotopes, e.g. ¹²⁵I, enzymes, antibodies, polynucleotides and linkers such as biotin. Labelled polypeptides may be used in diagnostic procedures such as immunoassays to determine the amount of a polypeptide in a sample. Polypeptides or labelled polypeptides may also be used in serological or cell-mediated immune assays for the detection of immune reactivity to said polypeptides in animals and humans using standard protocols.

GCR1/GCR2 polypeptides, variants, homologues, fragments and derivatives disclosed here, optionally labelled, my also be fixed to a solid phase, for example the surface of an immunoassay well or dipstick. Such labelled and/or immobilised polypeptides may be packaged into kits in a suitable container along with suitable reagents, controls, instructions and the like. Such polypeptides and kits may be used in methods of detection of antibodies to the polypeptides or their allelic or species variants by immunoassay.

Immunoassay methods are well known in the art and will generally comprise: (a) providing a polypeptide comprising an epitope bindable by an antibody against said protein; (b) incubating a biological sample with said polypeptide under conditions which allow for the formation of an antibody-antigen complex; and (c) determining whether antibody-antigen complex comprising said polypeptide is formed.

The GCR1/GCR2 polypeptides, variants, homologues, fragments and derivatives disclosed here may be used in in vitro or in vivo cell culture systems to study the role of their corresponding genes and homologues thereof in cell function, including their function in disease. For example, truncated or modified polypeptides may be introduced into a cell to disrupt the normal functions which occur in the cell. The polypeptides may be introduced into the cell by in situ expression of the polypeptide from a recombinant expression vector (see below). The expression vector optionally carries an inducible promoter to control the expression of the polypeptide.

The use of appropriate host cells, such as insect cells or mammalian cells, is expected to provide for such post-translational modifications (e.g. myristolation, glycosylation, truncation, lapidation and tyrosine, serine or threonine phosphorylation) as may be needed to confer optimal biological activity on recombinant expression products. Such cell culture systems in which the GCR1/GCR2 polypeptides, variants, homologues, fragments and derivatives disclosed here are expressed may be used in assay systems to identify candidate substances which interfere with or enhance the functions of the polypeptides in the cell.

GCR1/GCR2 Nucleic Acids

The methods and compositions described here provide generally for a number of GCR1 and GCR2 nucleic acids, together with fragments, homologues, variants and derivatives thereof. These nucleic acid sequences preferably encode the polypeptide sequences disclosed here, and particularly in the sequence listings. Preferably, the polynucleotides comprise Stella and/or Fragilis nucleic acids, preferably selected from the group consisting of: SEQ ID NO: 1, 3, 5, 6, 7, 8 or 9, fragments, homologues, variants and derivatives thereof.

In particular, we provide for nucleic acids which encode any of the GCR1 and/or GCR2 polypeptides disclosed here. Thus, the terms “GCR nucleic acid”, “GCR1 nucleic acid” and “GCR2 nucleic acid” should be construed accordingly. Preferably, however, such nucleic acids comprise any of the sequences set out as SEQ ID NO: 1, 3, 5, 6, 7, 8 or 9 or a sequence encoding any of the polypeptides SEQ ID NO: 2 and 4, and a fragment, homologue, variant or derivative of such a nucleic acid. The above terms therefore preferably should be taken to refer to these sequences.

As used here in this document, the terms “polynucleotide”, “nucleotide”, and nucleic acid are intended to be synonymous with each other. “Polynucleotide” generally refers to any polyribonucleotide or polydeoxribonucleotide, which may be unmodified RNA or DNA or modified RNA or DNA. “Polynucleotides” include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications has been made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short polynucleotides, often referred to as oligonucleotides.

It will be understood by a skilled person that numerous different polynucleotides and nucleic acids can encode the same polypeptide as a result of the degeneracy of the genetic code. In addition, it is to be understood that skilled persons may, using routine techniques, make nucleotide substitutions that do not affect the polypeptide sequence encoded by the polynucleotides described here to reflect the codon usage of any particular host organism in which the polypeptides are to be expressed.

Variants, Derivatives and Homologues

The polynucleotides described here may comprise DNA or RNA. They may be single-stranded or double-stranded. They may also be polynucleotides which include within them synthetic or modified nucleotides. A number of different types of modification to oligonucleotides are known in the art. These include methylphosphonate and phosphorothioate backbones, addition of acridine or polylysine chains at the 3′ and/or 5′ ends of the molecule. For the purposes of the present document, it is to be understood that the polynucleotides described herein may be modified by any method available in the art. Such modifications may be carried out in order to enhance the in vivo activity or life span of polynucleotides.

Where the polynucleotide is double-stranded, both strands of the duplex, either individually or in combination, are encompassed by the methods and compositions described here. Where the polynucleotide is single-stranded, it is to be understood that the complementary sequence of that polynucleotide is also included.

The terms “variant”, “homologue” or “derivative” in relation to a nucleotide sequence include any substitution of, variation of, modification of, replacement of, deletion of or addition of one (or more) nucleotides from or to the sequence providing the resultant nucleotide sequence is specific for pluripotent cells, preferably specific for PGCs, ES cells or EG cells. Most preferably, the resultant nucleotide sequence is specific for PGCs.

As indicated above, with respect to sequence identity, a “homologue” has preferably at least 5% identity, at least 10% identity, at least 15% identity, at least 20% identity, at least 25% identity, at least 30% identity, at least 35% identity, at least 40% identity, at least 45% identity, at least 50% identity, at least 55% identity, at least 60% identity, at least 65% identity, at least 70% identity, at least 75% identity, at least 80% identity, at least 85% identity, at least 90% identity, or at least 95% identity to the relevant sequence shown in the sequence listings.

More preferably there is at least 95% identity, more preferably at least 96% identity, more preferably at least 97% identity, more preferably at least 98% identity, more preferably at least 99% identity. Nucleotide homology comparisons may be conducted as described above. A preferred sequence comparison program is the GCG Wisconsin Bestfit program described above. The default scoring matrix has a match value of 10 for each identical nucleotide and −9 for each mismatch. The default gap creation penalty is −50 and the default gap extension penalty is −3 for each nucleotide.

Hybridisation

We further describe nucleotide sequences that are capable of hybridising selectively to any of the sequences presented herein, or any variant, fragment or derivative thereof, or to the complement of any of the above. Nucleotide sequences are preferably at least 15 nucleotides in length, more preferably at least 20, 30, 40 or 50 nucleotides in length.

The term “hybridisation” as used herein shall include “the process by which a strand of nucleic acid joins with a complementary strand through base pairing” as well as the process of amplification as carried out in polymerase chain reaction technologies.

Polynucleotides capable of selectively hybridising to the nucleotide sequences presented herein, or to their complement, will be generally at least 70%, preferably at least 80 or 90% and more preferably at least 95% or 98% homologous to the corresponding nucleotide sequences presented herein over a region of at least 20, preferably at least 25 or 30, for instance at least 40, 60 or 100 or more contiguous nucleotides.

The term “selectively hybridisable” means that the polynucleotide used as a probe is used under conditions where a target polynucleotide is found to hybridize to the probe at a level significantly above background. The background hybridization may occur because of other polynucleotides present, for example, in the cDNA or genomic DNA library being screening. In this event, background implies a level of signal generated by interaction between the probe and a non-specific DNA member of the library which is less than 10 fold, preferably less than 100 fold as intense as the specific interaction observed with the target DNA. The intensity of interaction may be measured, for example, by radiolabelling the probe, e.g. with ³²P.

Hybridisation conditions are based on the melting temperature (Tm) of the nucleic acid binding complex, as taught in Berger and Kimmel (1987, Guide to Molecular Cloning Techniques, Methods in Enzymology, Vol 152, Academic Press, San Diego Calif.), and confer a defined “stringency” as explained below.

Maximum stringency typically occurs at about Tm-5° C. (5° C. below the Tm of the probe); high stringency at about 5° C. to 10° C. below Tm; intermediate stringency at about 10° C. to 20° C. below Tm; and low stringency at about 20° C. to 25° C. below Tm. As will be understood by those of skill in the art, a maximum stringency hybridisation can be used to identify or detect identical polynucleotide sequences while an intermediate (or low) stringency hybridisation can be used to identify or detect similar or related polynucleotide sequences.

In a preferred aspect, we disclose nucleotide sequences that can hybridise to a GCR1/GCR2 nucleic acid, or a fragment, homologue, variant or derivative thereof, under stringent conditions (e.g. 65° C. and 0.1×SSC {1×SSC=0.15 M NaCl, 0.015 M Na₃Citrate pH 7.0}).

Where a polynucleotide is double-stranded, both strands of the duplex, either individually or in combination, are encompassed by the present disclosure. Where the polynucleotide is single-stranded, it is to be understood that the complementary sequence of that polynucleotide is also disclosed and encompassed.

Polynucleotides which are not 100% homologous to the sequences disclosed here but fall within the disclosure can be obtained in a number of ways. Other variants of the sequences described herein may be obtained for example by probing DNA libraries made from a range of individuals, for example individuals from different populations. In addition, other viral/bacterial, or cellular homologues particularly cellular homologues found in mammalian cells (e.g. rat, mouse, bovine and primate cells, including human cells), may be obtained and such homologues and fragments thereof in general will be capable of selectively hybridising to the sequences shown in the sequence listing herein. Such sequences may be obtained by probing cDNA libraries made from or genomic DNA libraries from other animal species, and probing such libraries with probes comprising all or part of SEQ ID NOs: 1 or 3 under conditions of medium to high stringency. Similar considerations apply to obtaining species homologues and allelic variants of GCR1 and GCR2.

The polynucleotides described here may be used to produce a primer, e.g. a PCR primer, a primer for an alternative amplification reaction, a probe e.g. labelled with a revealing label by conventional means using radioactive or non-radioactive labels, or the polynucleotides may be cloned into vectors. Such primers, probes and other fragments will be at least 15, preferably at least 20, for example at least 25, 30 or 40 nucleotides in length, and are also encompassed by the term polynucleotides as used herein. Preferred fragments are less than 500, 200, 100, 50 or 20 nucleotides in length.

Polynucleotides such as a DNA polynucleotides and probes may be produced recombinantly, synthetically, or by any means available to those of skill in the art. They may also be cloned by standard techniques.

In general, primers will be produced by synthetic means, involving a step wise manufacture of the desired nucleic acid sequence one nucleotide at a time. Techniques for accomplishing this using automated techniques are readily available in the art.

Longer polynucleotides will generally be produced using recombinant means, for example using PCR polymerase chain reaction) cloning techniques. This will involve making a pair of primers (e.g. of about 15 to 30 nucleotides) flanking a region of the sequence which it is desired to clone, bringing the primers into contact with mRNA or cDNA obtained from an animal or human cell, performing a polymerase chain reaction under conditions which bring about amplification of the desired region, isolating the amplified fragment (e.g. by purifying the reaction mixture on an agarose gel) and recovering the amplified DNA. The primers may be designed to contain suitable restriction enzyme recognition sites so that the amplified DNA can be cloned into a suitable cloning vector

Nucleotide Vectors

The polynucleotides can be incorporated into a recombinant replicable vector. The vector may be used to replicate the nucleic acid in a compatible host cell. Thus in a further embodiment, we provide a method of making polynucleotides by introducing a polynucleotide into a replicable vector, introducing the vector into a compatible host cell, and growing the host cell under conditions which bring about replication of the vector. The vector may be recovered from the host cell. Suitable host cells include bacteria such as E. coli, yeast, mammalian cell lines and other eukaryotic cell lines, for example insect Sf9 cells.

Preferably, a polynucleotide in a vector is operably linked to a control sequence that is capable of providing for the expression of the coding sequence by the host cell, i.e. the vector is an expression vector. The term “operably linked” means that the components described are in a relationship permitting them to function in their intended manner. A regulatory sequence “operably linked” to a coding sequence is ligated in such a way that expression of the coding sequence is achieved under condition compatible with the control sequences.

The control sequences may be modified, for example by the addition of further transcriptional regulatory elements to make the level of transcription directed by the control sequences more responsive to transcriptional modulators.

Vectors may be transformed or transfected into a suitable host cell as described below to provide for expression of a protein. This process may comprise culturing a host cell transformed with an expression vector as described above under conditions to provide for expression by the vector of a coding sequence encoding the protein, and optionally recovering the expressed protein.

The vectors may be for example, plasmid or virus vectors provided with an origin of replication, optionally a promoter for the expression of the said polynucleotide and optionally a regulator of the promoter. The vectors may contain one or more selectable marker genes, for example an ampicillin resistance gene in the case of a bacterial plasmid or a neomycin resistance gene for a mammalian vector. Vectors may be used, for example, to transfect or transform a host cell.

Control sequences operably linked to sequences encoding the protein include promoters/enhancers and other expression regulation signals. These control sequences may be selected to be compatible with the host cell for which the expression vector is designed to be used in. The term “promoter” is well-known in the art and encompasses nucleic acid regions ranging in size and complexity from minimal promoters to promoters including upstream elements and enhancers.

The promoter is typically selected from promoters which are functional in mammalian cells, although prokaryotic promoters and promoters functional in other eukaryotic cells may be used. The promoter is typically derived from promoter sequences of viral or eukaryotic genes. For example, it may be a promoter derived from the genome of a cell in which expression is to occur. With respect to eukaryotic promoters, they may be promoters that function in a ubiquitous manner (such as promoters of α-actin, β-actin, tubulin) or, alternatively, a tissue-specific manner (such as promoters of the genes for pyruvate kinase). They may also be promoters that respond to specific stimuli, for example promoters that bind steroid hormone receptors. Viral promoters may also be used, for example the Moloney murine leukaemia virus long terminal repeat (MMLV LTR) promoter, the Rous sarcoma virus (RSV) LTR promoter or the human cytomegalovirus (CMV) IE promoter.

It may also be advantageous for the promoters to be inducible so that the levels of expression of the heterologous gene can be regulated during the life-time of the cell. Inducible means that the levels of expression obtained using the promoter can be regulated.

In addition, any of these promoters may be modified by the addition of further regulatory sequences, for example enhancer sequences. Chimeric promoters may also be used comprising sequence elements from two or more different promoters described above.

Host Cells

Vectors and polynucleotides disclosed here may be introduced into host cells for the purpose of replicating the vectors/polynucleotides and/or expressing the proteins. Although the proteins may be produced using prokaryotic cells as host cells, it is preferred to use eukaryotic cells, for example yeast, insect or mammalian cells, in particular mammalian cells.

Vectors/polynucleotides may introduced into suitable host cells using a variety of techniques known in the art, such as transfection, transformation and electroporation. Where vectors/polynucleotides as disclosed here are to be administered to animals, several techniques are known in the art, for example infection with recombinant viral vectors such as retroviruses, herpes simplex viruses and adenoviruses, direct injection of nucleic acids and biolistic transformation.

Protein Expression and Purification

Host cells comprising polynucleotides disclosed here may be used to express proteins. Host cells may be cultured under suitable conditions which allow expression of the proteins. Expression of the proteins described here may be constitutive such that they are continually produced, or inducible, requiring a stimulus to initiate expression. In the case of inducible expression, protein production can be initiated when required by, for example, addition of an inducer substance to the culture medium, for example dexamethasone or IPTG.

Proteins can be extracted from host cells by a variety of techniques known in the art, including enzymatic, chemical and/or osmotic lysis and physical disruption.

Recombinant Stella and Fragilis Proteins

Nucleotide sequences of Stella and Fragilis are cloned into a TRI-system vector (Qiagen). Stella sequence comprising the second codon onwards (i.e., an N terminal fragment of Stella without the first ATG codon) is cloned into a pQE vector using appropriate restriction enzyme sites, and according to the manufacturers instructions. QIAexpress pQE vectors enable high-level expression of 6×His-tagged proteins in E. coli.

A His tag is placed in the N terminal portion of the Stella gene. Recombinant protein is purified by affinity chromatography on a Ni-NTA column, according to manufacturer's instructions. The His tag is cleaved using a suitable protease.

Recombinantly expressed Stella and Fragilis protein are found to be biologically active.

Antibodies

Antibodies, as used herein, refers to complete antibodies or antibody fragments capable of binding to a selected target, and including Fv, ScFv, Fab′ and F(ab′)₂, monoclonal and polyclonal antibodies, engineered antibodies including chimeric, CDR-grafted and humanised antibodies, and artificially selected antibodies produced using phage display or alternative techniques. Small fragments, such as Fv and ScFv, possess advantageous properties for diagnostic and therapeutic applications on account of their small size and consequent superior tissue distribution.

The antibodies according described here are especially indicated for the detection of PGCs and other pluripotent cells, such as ES or EG cells. Accordingly, they may be altered antibodies comprising an effector protein such as a label. Especially preferred are labels which allow the imaging of the distribution of the antibody in vivo or in vitro. Such labels may be radioactive labels or radioopaque labels, such as metal particles, which are readily visualisable within an embryo or a cell mass. Moreover, they may be fluorescent labels or other labels which are visualisable on tissue samples.

Recombinant DNA technology may be used to improve the antibodies as described here. Thus, chimeric antibodies may be constructed in order to decrease the immunogenicity thereof in diagnostic or therapeutic applications. Moreover, immunogenicity may be minimised by humanising the antibodies by CDR grafting [see European Patent Application 0 239 400 (Winter)] and, optionally, framework modification [EP 0 239 400].

Antibodies may be obtained from animal serum, or, in the case of monoclonal antibodies or fragments thereof, produced in cell culture. Recombinant DNA technology may be used to produce the antibodies according to established procedure, in bacterial or preferably mammalian cell culture. The selected cell culture system preferably secretes the antibody product.

Therefore, we disclose a process for the production of an antibody comprising culturing a host, e.g. E. coli or a mammalian cell, which has been transformed with a hybrid vector comprising an expression cassette comprising a promoter operably linked to a first DNA sequence encoding a signal peptide linked in the proper reading frame to a second DNA sequence encoding said antibody protein, and isolating said protein.

Multiplication of hybridoma cells or mammalian host cells in vitro is carried out in suitable culture media, which are the customary standard culture media, for example Dulbecco's Modified Eagle Medium (DMEM) or RPMI 1640 medium, optionally replenished by a mammalian serum, e.g. foetal calf serum, or trace elements and growth sustaining supplements, e.g. feeder cells such as normal mouse peritoneal exudate cells, spleen cells, bone marrow macrophages, 2-aminoethanol, insulin, transferrin, low density lipoprotein, oleic acid, or the like. Multiplication of host cells which are bacterial cells or yeast cells is likewise carried out in suitable culture media known in the art, for example for bacteria in medium LB, NZCYM, NZYM, NZM, Terrific Broth, SOB, SOC, 2×YT, or M9 Minimal Medium, and for yeast in medium YPD, YEPD, Minimal Medium, or Complete Minimal Dropout Medium.

In vitro production provides relatively pure antibody preparations and allows scale-up to give large amounts of the desired antibodies. Techniques for bacterial cell, yeast or mammalian cell cultivation are known in the art and include homogeneous suspension culture, e.g. in an airlift reactor or in a continuous stirrer reactor, or immobilised or entrapped cell culture, e.g. in hollow fibres, microcapsules, on agarose microbeads or ceramic cartridges.

Large quantities of the desired antibodies can also be obtained by multiplying mammalian cells in vivo. For this purpose, hybridoma cells producing the desired antibodies are injected into histocompatible mammals to cause growth of antibody-producing tumours. Optionally, the animals are primed with a hydrocarbon, especially mineral oils such as pristane (tetramethyl-pentadecane), prior to the injection. After one to three weeks, the antibodies are isolated from the body fluids of those mammals. For example, hybridoma cells obtained by fusion of suitable myeloma cells with antibody-producing spleen cells from Balb/c mice, or transfected cells derived from hybridoma cell line Sp2/0 that produce the desired antibodies are injected intraperitoneally into Balb/c mice optionally pre-treated with pristane, and, after one to two weeks, ascitic fluid is taken from the animals.

The foregoing, and other, techniques are discussed in, for example, Kohler and Milstein, (1975) Nature 256:495-497; U.S. Pat. No. 4,376,110; Harlow and Lane, Antibodies: a Laboratory Manual, (1988) Cold Spring Harbor, incorporated herein by reference. Techniques for the preparation of recombinant antibody molecules is described in the above references and also in, for example, EP 0623679; EP 0368684 and EP 0436597, which are incorporated herein by reference.

The cell culture supernatants are screened for the desired antibodies, preferentially by immunofluorescent staining of PGCs or other pluripotent cells, such as ES or EG cells, by immunoblotting, by an enzyme immunoassay, e.g. a sandwich assay or a dot-assay, or a radioimmunoassay.

For isolation of the antibodies, the immunoglobulins in the culture supernatants or in the ascitic fluid may be concentrated, e.g. by precipitation with ammonium sulphate, dialysis against hygroscopic material such as polyethylene glycol, filtration through selective membranes, or the like. If necessary and/or desired, the antibodies are purified by the customary chromatography methods, for example gel filtration, ion-exchange chromatography, chromatography over DEAE-cellulose and/or (immuno-) affinity chromatography, e.g. affinity chromatography with GCR1 or GCR2, or fragments thereof, or with Protein-A.

Hybridoma cells secreting the monoclonal antibodies are also provided. Preferred hybridoma cells are genetically stable, secrete monoclonal antibodies of the desired specificity and can be activated from deep-frozen cultures by thawing and recloning.

Also included is a process for the preparation of a hybridoma cell line secreting monoclonal antibodies directed to GCR1 and/or GCR2, characterised in that a suitable mammal, for example a Balb/c mouse, is immunised with a one or more GCR1 or GCR2 polypeptides, or antigenic fragments thereof; antibody-producing cells of the immunised mammal are fused with cells of a suitable myeloma cell line, the hybrid cells obtained in the fusion are cloned, and cell clones secreting the desired antibodies are selected. For example spleen cells of Balb/c mice immunised with GCR1 and/or GCR2 are fused with cells of the myeloma cell line PAI or the myeloma cell line Sp2/0-Ag14, the obtained hybrid cells are screened for secretion of the desired antibodies, and positive hybridoma cells are cloned.

Preferred is a process for the preparation of a hybridoma cell line, characterised in that Balb/c mice are immunised by injecting subcutaneously and/or intraperitoneally between 10 and 10⁷ and 10⁸ cells expressing GCR1 and/or GCR2 and a suitable adjuvant several times, e.g. four to six times, over several months, e.g. between two and four months, and spleen cells from the immunised mice are taken two to four days after the last injection and fused with cells of the myeloma cell line PAI in the presence of a fusion promoter, preferably polyethylene glycol. Preferably the myeloma cells are fused with a three- to twentyfold excess of spleen cells from the immunised mice in a solution containing about 30% to about 50% polyethylene glycol of a molecular weight around 4000. After the fusion the cells are expanded in suitable culture media as described hereinbefore, supplemented with a selection medium, for example HAT medium, at regular intervals in order to prevent normal myeloma cells from overgrowing the desired hybridoma cells.

Recombinant DNAs comprising an insert coding for a heavy chain variable domain and/or for a light chain variable domain of antibodies directed to GCR1 and/or GCR2 as described hereinbefore are also disclosed. By definition such DNAs comprise coding single stranded DNAs, double stranded DNAs consisting of said coding DNAs and of complementary DNAs thereto, or these complementary (single stranded) DNAs themselves.

Furthermore, DNA encoding a heavy chain variable domain and/or for a light chain variable domain of antibodies directed to GCR1 and/or GCR2 can be enzymatically or chemically synthesised DNA having the authentic DNA sequence coding for a heavy chain variable domain and/or for the light chain variable domain, or a mutant thereof. A mutant of the authentic DNA is a DNA encoding a heavy chain variable domain and/or a light chain variable domain of the above-mentioned antibodies in which one or more amino acids are deleted or exchanged with one or more other amino acids. Preferably said modification(s) are outside the CDRs of the heavy chain variable domain and/or of the light chain variable domain of the antibody. Such a mutant DNA is also intended to be a silent mutant wherein one or more nucleotides are replaced by other nucleotides with the new codons coding for the same amino acid(s). Such a mutant sequence is also a degenerated sequence. Degenerated sequences are degenerated within the meaning of the genetic code in that an unlimited number of nucleotides are replaced by other nucleotides without resulting in a change of the amino acid sequence originally encoded. Such degenerated sequences may be useful due to their different restriction sites and/or frequency of particular codons which are preferred by the specific host, particularly E. coli, to obtain an optimal expression of the heavy chain murine variable domain and/or a light chain murine variable domain.

The term mutant is intended to include a DNA mutant obtained by in vitro mutagenesis of the authentic DNA according to methods known in the art.

For the assembly of complete tetrameric immunoglobulin molecules and the expression of chimeric antibodies, the recombinant DNA inserts coding for heavy and light chain variable domains are fused with the corresponding DNAs coding for heavy and light chain constant domains, then transferred into appropriate host cells, for example after incorporation into hybrid vectors.

Also disclosed are recombinant DNAs comprising an insert coding for a heavy chain murine variable domain of an antibody directed to GCR1 and/or GCR2 fused to a human constant domain g, for example γ1, γ2, γ3 or γ4, preferably γ1 or γ4. Likewise the invention concerns recombinant DNAs comprising an insert coding for a light chain murine variable domain of an antibody directed to GCR1 and/or GCR2 fused to a human constant domain κ or λ, preferably κ.

In another embodiment, we disclose recombinant DNAs coding for a recombinant polypeptide wherein the heavy chain variable domain and the light chain variable domain are linked by way of a spacer group, optionally comprising a signal sequence facilitating the processing of the antibody in the host cell and/or a DNA coding for a peptide facilitating the purification of the antibody and/or a cleavage site and/or a peptide spacer and/or an effector molecule.

The DNA coding for an effector molecule is intended to be a DNA coding for the effector molecules useful in diagnostic or therapeutic applications. Thus, effector molecules which are toxins or enzymes, especially enzymes capable of catalysing the activation of prodrugs, are particularly indicated. The DNA encoding such an effector molecule has the sequence of a naturally occurring enzyme or toxin encoding DNA, or a mutant thereof, and can be prepared by methods well known in the art.

Anti-Peptide Stella and Fragilis Antibodies

Anti-peptide antibodies are produced against Stella and Fragilis peptide sequences. The sequences chosen are as follow:

GCR1 (Fragilis):
ASGGQPPNYERIKEEYE and RDRKMVGDVTGAQAYA
GCR2 (Stella):
MEEPSEKVDPMKDPET and CHYQRWDPSENAKIGKN

Antibodies are produced by injection into rabbits, and other conventional means, as described in for example, Harlow and Lane (supra).

Antibodies are checked by Elisa assay and by Western blotting, and used for immunostaining as described in the Examples.

Detection of Pluripotent Cells in Cells Populations

Polynucleotide probes or antibodies as described here may be used for the detection of pluripotent cells such as primordial germ cells (PGCs), stem cells such as embryonic stem (ES) and embryonic germ (EG) cells in cell populations. As used herein, a “cell population” is any collection of cells which may contain one or more PGCs, ES or EG cells. Preferably, the collection of cells does not consist solely of PGCs, but comprises at least one other cell type.

Cell populations comprise embryos and embryo tissue, but also adult tissues and tissues grown in culture and cell preparations derived from any of the foregoing.

Polynucleotides as described here may be used for detection of GCR1 and GCR2 transcripts in PGCs or other pluripotent cells, such as ES or EG cells, by nucleic acid hybridisation techniques. Such techniques include PCR, in which primers are hybridised to GCR1 and/or GCR2 transcripts and used to amplify the transcripts, to provide a detectable signal; and hybridisation of labelled probes, in which probes specific for an unique sequence in the GCR1 and/or GCR2 transcript are used to detect the transcript in the target cells.

As noted hereinbefore, probes may be labelled with radioactive, radioopaque, fluorescent or other labels, as is known in the art.

The antibodies may also be used to detect GCR1 and/or GCR2. GRC1, in particular, possesses an extracellular domain which may be targeted by an anti-GCR1 antibody and detected at the cell surface. Alternatively, intracellular scFv may be used to detect GCR1 and/or GCR2 within the cell.

Particularly indicated are immunostaining and FACS techniques. Suitable fluorophores are known in the art, and include chemical fluorophores and fluorescent polypeptides, such as GFP and mutants thereof (see WO 97/28261). Chemical fluorophores may be attached to immunoglobulin molecules by incorporating binding sites therefor into the immunoglobulin molecule during the synthesis thereof.

Preferably, the fluorophore is a fluorescent protein, which is advantageously GFP or a mutant thereof. GFP and its mutants may be synthesised together with the immunoglobulin or target molecule by expression therewith as a fusion polypeptide, according to methods well known in the art. For example, a transcription unit may be constructed as an in-frame fusion of the desired GFP and the immunoglobulin or target, and inserted into a vector as described above, using conventional PCR cloning and ligation techniques.

Antibodies may be labelled with any label capable of generating a signal. The signal may be any detectable signal, such as the induction of the expression of a detectable gene product. Examples of detectable gene products include bioluminescent polypeptides, such as luciferase and GFP, polypeptides detectable by specific assays, such as β-galactosidase and CAT, and polypeptides which modulate the growth characteristics of the host cell, such as enzymes required for metabolism such as HIS3, or antibiotic resistance genes such as G418. In a preferred aspect, the signal is detectable at the cell surface. For example, the signal may be a luminescent or fluorescent signal, which is detectable from outside the cell and allows cell sorting by FACS or other optical sorting techniques.

Preferred is the use of optical immunosensor technology, based on optical detection of fluorescently-labelled antibodies. Immunosensors are biochemical detectors comprising an antigen or antibody species coupled to a signal transducer which detects the binding of the complementary species (Rabbany et al., 1994 Crit Rev Biomed Eng 22:307-346; Morgan et al., 1996 Clin Chem 42:193-209). Examples of such complementary species include the antigen Zif 268 and the anti-Zif 268 antibody. Immunosensors produce a quantitative measure of the amount of antibody, antigen or hapten present in a complex sample such as serum or whole blood (Robinson 1991 Biosens Bioelectron 6:183-191). The sensitivity of immunosensors makes them ideal for situations requiring speed and accuracy (Rabbany et al., 1994 Crit Rev Biomed Eng 22:307-346).

Detection techniques employed by immunosensors include electrochemical, piezoelectric or optical detection of the immunointeraction (Ghindilis et al., 1998 Biosens Bioelectron 1:113-131). An indirect immunosensor uses a separate labelled species that is detected after binding by, for example, fluorescence or luminescence (Morgan et al., 1996 Clin Chem 42:193-209). Direct immunosensors detect the binding by a change in potential difference, current, resistance, mass, heat or optical properties (Morgan et al., 1996 Clin Chem 42:193-209). Indirect immunosensors may encounter fewer problems due to non-specific binding (Attridge et al., 1991 Biosens Bioelecton 6:201-214; Morgan et al., 1996 Clin Chem 42:193-209).

Further Aspects of the Invention

We provide a nucleic acid molecule which is at least 90% homologous to SEQ ID NO: 1 and a nucleic acid molecule which is at least 75% homologous to SEQ ID NO: 3.

We disclose polynucleotides which comprise a contiguous stretch of nucleotides from SEQ ID NO: 1 or SEQ ID NO: 3, or any of SEQ ID NOS: 5 to 9, or of a sequence at least 90% homologous thereto. Advantageously, this stretch of contiguous nucleotides is 50 nucleotides in length, preferably 40, 35, 30, 25, 20, 15 or 10 nucleotides in length.

The genes GCR1 and GCR2 encode novel polypeptides, the sequences of which are set forth in SEQ ID NO: 2 and SEQ ID NO: 4. We therefore disclose polypeptides encoded by the nucleic acids described here. Preferably, the polypeptides have the sequences set forth in SEQ ID NO: 2 and SEQ ID NO: 4.

Moreover, we provide a method by which genes specifically expressed in PGCs or other pluripotent cells, such as ES or EG cells, may be isolated, comprising the steps of: (a) providing a population of cells containing PGCs or other pluripotent cells, such as ES or EG cells; (b) isolating one or more PGCs or other pluripotent cells, such as ES or EG cells, therefrom and providing single-cell isolates; (c) amplifying the transcribed nucleic acid present in a single cell; (d) conducting a subtractive hybridisation screen to identify transcripts present in the PGCs or other pluripotent cells, such as ES or EG cells, but not in somatic cells; and (e) probing a nucleic acid library with one or more transcripts identified in d) to clone one or more genes which are specifically expressed.

Further aspects of the invention are now set out in the following numbered paragraphs; it is to be understood that the invention encompasses these aspects:

Paragraph 1. A nucleic acid having at least 90% homology with the sequence set forth in SEQ. ID. No. 1.

Paragraph 2. A nucleic acid having at least 75% homology with the sequence set forth in SEQ. ID. No. 3.

Paragraph 3. A nucleic acid comprising a sequence of 25 contiguous nucleotides of the nucleic acid of Paragraph 1 or Paragraph 2.

Paragraph 4. A nucleic acid comprising a sequence of 15 contiguous nucleotides of the nucleic acid of Paragraph 1 or Paragraph 2.

Paragraph 5. The complement of a nucleic acid sequence according to any preceding Paragraph.

Paragraph 6. A nucleic acid according to any one of Paragraphs 1 to 5, comprising one or more nucleotide substitutions, wherein such substitutions do not alter the coding specificity of said nucleic acid as a result of the degeneracy of the genetic code.

Paragraph 7. A polypeptide encoded by a nucleic acid according to any preceding Paragraph.

Paragraph 8. A method for identifying a primordial germ cell in a population of cells, comprising detecting the expression of a nucleic acid sequence according to Paragraph 1 or Paragraph 2, or a homologue thereof.

Paragraph 9. A method according to Paragraph 8, comprising the steps of amplifying nucleic acids from putative PGCs using 5′ and 3′ primers specific for GCR1 and/or GCR2, and detecting amplified nucleic acid thus produced.

Paragraph 10. A method according to Paragraph 8, wherein the expression of the nucleic acid sequence is detected by in situ hybridisation.

Paragraph 11. A method according to Paragraph 8, wherein the expression of the nucleic acid sequence is determined by detecting the protein product encoded thereby.

Paragraph 12. A method according to Paragraph 11, wherein the protein product is detected by immunostaining.

Paragraph 13. An antibody specific for a polypeptide according to Paragraph 7.

Paragraph 14. An antibody according to Paragraph 13, specific for the extracellular domain of GCR1.

Paragraph 15. Use of an antibody according to Paragraph 13 or Paragraph 14 for the identification of a PGC in a population of cells.

Paragraph 16. A PGC when identified by a method according to any one of Paragraphs 8 to 12.

Paragraph 17. A method for isolating a gene specifically expressed in PGCs, comprising the steps of: a) providing a population of cells containing PGCs; b)isolating one or more PGCs therefrom and providing single-cell PGC isolates; c) amplifying the transcribed nucleic acid present in a single PGC; d) conducting a subtractive hybridization screen to identify transcripts present in PGCs but not is somatic cells; and e) probing a nucleic acid library with one or more transcripts identified in d) to clone one or more genes which are specifically expressed in PGCs.

Paragraph 18. A GCR1 polypeptide, or a fragment, homologue, variant or derivative thereof.

Paragraph 19. A polypeptide according to paragraph 18, which has at least 50%, 60%, 70%, 80%, 90% or 95% homology to a sequence shown in SEQ ID NO: 2.

Paragraph 20. A GCR2 polypeptide, or a fragment, homologue, variant or derivative thereof.

Paragraph 21. A polypeptide according to paragraph 20, which has at least 50%, 60%, 70%, 80%, 90% or 95% homology to a sequence shown in SEQ ID NO: 4.

Paragraph 22. A nucleic acid encoding a polypeptide according to any preceding paragraph.

Paragraph 23. A nucleic acid having at least 90% homology with the sequence set forth in SEQ ID NO: 1, or a fragment, variant or derivative thereof.

Paragraph 24. A nucleic acid having at least 75% homology with the sequence set forth in SEQ ID NO: 3, SEQ ID NO: 5, SEQ ID NO: 6, SEQ ID NO: 7, SEQ ID NO: 8 or SEQ ID NO: 9 or a fragment, variant or derivative thereof.

Paragraph 25. A nucleic acid comprising a sequence of 25 contiguous nucleotides of a nucleic acid according to paragraph 22, 23 or 24.

Paragraph 26. A nucleic acid comprising a sequence of 15 contiguous nucleotides of a nucleic acid according to any of paragraphs 22 to 25.

Paragraph 27. The complement of a nucleic acid sequence according to any of paragraphs 22 to 26.

Paragraph 28. A nucleic acid according to any of paragraphs 22 to 27, comprising one or more nucleotide substitutions, wherein such substitutions do not alter the coding specificity of said nucleic acid as a result of the degeneracy of the genetic code.

Paragraph 29. A polypeptide encoded by a nucleic acid according to any preceding paragraph.

Paragraph 30. A polypeptide according to paragraph 29, in which the polypeptide comprises a sequence shown in SEQ ID NO: 2 or SEQ ID NO: 4.

Paragraph 31. A method for identifying a pluripotent cell, comprising detecting the presence of a polypeptide according to any of paragraphs 18 to 21, 29 or 30 or the expression of a nucleic acid according to any of paragraphs 22 to 28, or a homologue thereof.

Paragraph 32. A method according to paragraph 31, comprising the steps of amplifying nucleic acids from a putative pluripotent cell using 5′ and 3′ primers specific for GCR1 and/or GCR2, and detecting amplified nucleic acid thus produced.

Paragraph 33. A method according to paragraph 31, wherein the expression of the nucleic acid sequence is detected by in situ hybridisation.

Paragraph 34. A method according to paragraph 25, wherein the expression of the nucleic acid sequence is determined by detecting the protein product encoded thereby.

Paragraph 35. A method according to paragraph 31 or paragraph 34, wherein the protein product is detected by immunostaining.

Paragraph 36. An antibody specific for a polypeptide according to any of paragraphs 18 to 21, 29 or 30.

Paragraph 37. An antibody according to paragraph 36, which is capable of specifically binding to an extracellular domain of GCR1.

Paragraph 38. Use of an antibody according to paragraph 36 or paragraph 37 for the identification and/or isolation of a pluripotent cell.

Paragraph 39. A pluripotent cell identified by a method according to any one of paragraphs 31 to 35 and 38.

Paragraph 40. A method for isolating a gene specifically expressed in a pluripotent cell, comprising the steps of (a) providing a population of cells containing a pluripotent cell; (b) isolating one or more pluripotent cells therefrom and providing single-cell pluripotent cell isolates; (c) amplifying the transcribed nucleic acid present in a single pluripotent cell; (d) conducting a subtractive hybridisation screen to identify transcripts present in pluripotent cells but not in somatic cells; and (e) probing a nucleic acid library with one or more transcripts identified in (d) to clone one or more genes which are specifically expressed in pluripotent cells.

Paragraph 41. A method according to any of paragraphs 31 to 35 or 40, a use according to paragraph 38, a pluripotent cell according to paragraph 40, in which the pluripotent cell is selected from the group consisting of a primordial germ cell (PGC), an embryonic stem cell (ES) and an embryonic germ cell (EG).

### EXAMPLES

### Example 1

### Identification of Genes Specific to the Earliest Population of Primordial Germ Cells (PGCs) by Single Cell cDNA Differential Screening

A method for single cell analysis is developed to identify genes that are involved in the specification of the germ cell lineage, which results in the establishment of a founder population of Priomordial Germ Cells (PGCs). It is determined that the lineage specification of PGCs accompanies the expression of a unique set of genes, which are not expressed in somatic cells.

The method for the identification of the genes is mainly based on the differential screening of the libraries made from single cells from day 7.25 mouse embryonic fragments that contain PGCs. The single cell cDNA differential screen was originally described by Brady and Iscove (1993), and subsequently modified by Cathaline Dulac and Richard Axel which resulted in the successful identification of the pheromone receptor genes from rat (Dulac, C. and Axel, 1995). The method of Axel's group is employed, with slight modifications as described.

Construction of Single Cell cDNAs from Embryonic Fragment Bearing the Earlies Population of PGCs

In the mouse, the earliest population of the PGCs is reported to consist of alkaline phosphatase positive cluster of some 40 cells, at the base of the emerging allantois at day 7.25 of gestation (Ginsburg, M. Snow, M. H. L., and McLaren, A. (1990)). The precise location of the PGC cluster in the inbred 129Sv and C57BL/6 strain is determined by microscopy using both whole-mount alkaline phosphatase staining and semi-thin sections stained by methylene blue. The earliest stage at which a cluster of PGCs can be detected is at the Late Streak stage (Downs, K. M., and Davies, T. (1993)), when a distinctively stained population of cells is found just beneath an epithelial lining from which the allantoic bud appears. This region is at the border between the extraembryonic and embryonic tissues just posterior to and above the most proximal part of the primitive streak. The cluster persists at this position at least until Early/Mid Bud stage. In the inbred 129Sv strain, the PGC cluster is found to contain a slightly larger number of the cells, which are more tightly packaged than in the C57BL/6 strain. The 129Sv strain is used for subsequent experiments, as a better recovery of the earliest PGCs is obtained.

129Sv embryos are isolated at E7.5 in DMEM plus 10% FCS buffered with 25 mM HEPES at room temperature and the developmental stage of each embryo is determined under a dissection microscope. The precise developmental stage can differ substantially even amongst embryos within the same litter. Embryos that are at the no bud or early bud (allantoic) stage are chosen for further dissection, which in part is dictated by the ease of identification of the region containing PGCs as seen under the dissection microscope. The fragment that is expected to contain the PGC cluster is cut out very precisely by means of solid glass needles. This region is dissociated it into single cells using 0.25% trypsin-1 mM EGTA/PBS treatment at 37° C. for 10 min, followed by gentle pipetting with a mouth pipette. The dissected fragment usually contained between 250-300 cells. The procedure for cell dispersal with this gentle procedure left the visceral endoderm layer remained as an intact cellular sheet.

We picked single cells randomly from the cell suspension by a mouth pipette and put individual single cells (but avoiding generating air bubbles), into a thin-walled PCR tube containing 4 μl of ice-cold cell lysis buffer (50 mM Tris-HCl pH8.3, 75 mM KCl, 3 mM MgCl₂, 0.5% NP-40, containing 80 ng/ml pd(T)24, 5 μg/ml prime RNase inhibitor, 324U/ml RNA guard, and 10 mM each of dATP, dCTP, dGTP, and dTTP). The volume of medium carried with the single cell is less than 0.5 μl. The tube is briefly centrifuged to ensure that the cell is indeed in the lysis buffer. During each separate experiment, we picked a total of 19 single cells, and left one tube without a cell, to serve as a negative control for the PCR amplification procedure. All the cells that are collected in tubes are kept on ice before starting the subsequent procedure.

The cells are lysed by incubating the tubes at 65° C. for 1 min, and then kept at room temperature for 1-2 min to allow the oligo dT to anneal the to RNA. First-strand cDNA synthesis is initiated by adding 50U of Moloney murine leukaemia virus (MMLV) and 0.5U of avian myeloblastosis virus (AMV) reverse transcriptase followed by incubation for 15 min at 37° C. The reverse transcriptases are inactivated for 10 min at 65° C. This reverse transcription reaction is restricted to 15 min, which allows the synthesis of relatively uniform size cDNAs of between 500 base-1000 bases in length from the C termini. This enables the subsequent PCR amplification to be fairly representative.

Next, in order to add the poly A tail to the 5 prime end of the synthesised first-strand cDNA, 4.5 μl of 2× tailing buffer (200 mM potassium cacodylate pH7.2, 4 mM CoCl₂, 0.4 mM DTT, 200 mM dATP containing 10 U of terminal transferase) is added to the reaction followed by incubation for 15 min at 37° C. The samples are heat inactivated for 10 min at 65° C. The reaction now contained synthesised cDNAs bearing poly T tail at their C termini and poly A stretch at their N termini, ready for the amplification by the PCR using the specific primer.

The contents of each tube is brought to 100 μl with a solution made of 10 mM Tris-HCl pH8.3, 50 mM KCl, 2.5 mM MgCl₂, 100 μg/ml bovine serum albumin, 0.05% Triton-X 100, 1 mM of dATP, dCTP, dGTP, dTTP, 10U of Taq polymerase, and 5 μg of the AL1 primer. The AL1 sequence is ATT GGA TCC AGG CCG CTC TGG ACA AAA TAT GAA TCC (T)₂₄. The PCR amplification is performed according to the following schedule: 94° C. for 1 min, 42° C. for 2 min, and 72° C. for 6 min with 10 s extension per cycle for 25 cycles. Five additional units of Taq polymerase are added before performing 25 more cycles with the same programme but without the extension time. Each tube at this point contains amplified cDNA products derived from a single cell. The protein contents of the solution are extracted by phenol/chloroform treatment, and the amplified cDNAs are precipitated by ethanol and eventually suspended in 100 μl of TE pH8.0. 5 μl of the cDNA solution is run on a 1.5% agarose gel to check the success of the amplification. Most of the samples show a very intense ‘smeared’ band ranging mainly between 500 bp to 1200 bp, indicating the efficient amplification of the single cell cDNA. Only the successfully amplified samples are used for the subsequent ‘cell typing’ analysis.

### Example 2

### Identification of PGCs by Examination of the Expression of Marker Genes

The embryonic fragment which is excised theoretically contains three major components: the allantoic mesoderm, PGCs, and extraembryonic mesoderm surrounding PGCs. In order to identify the single cell cDNA of PGC origin amongst these samples, positive and negative selection of the constructed cDNAs is performed, by examining the expression of four marker genes (BMP4, TNAP, Hoxb1, and Oct4), which are known to be either expressed or repressed in various cell types in this region.

At the No/Early Bud stage, BMP4 is reported to be expressed in the emerging allantois and mesodermal components of the developing amnion, chorion, and visceral yolk sac (Lawson, K. A., Dunn, N. R., Roelen, B. A. J., Zeinstra, L. M., Davis, A. M., Wright, C. V. E., Korving, J. P. W. F. M., and Hogan, B. L. M. (1999)). The boundary of BMP4 expression is very sharp, and the expression is completely excluded in the mesodermal region beneath the epithelial lining continuous from the amnionic mesoderm where the putative PGCs are determined. Therefore, BMP4 is used as a negative marker for the selection. Primer pairs are designed for amplifying the C terminal portion of BMP4 (5′:GCC ATA CCT TGA CCC GCA GAA G. 3′: AAA TGG CAC TCA GTT CAG TGG G). The PCR amplification is performed using 0.5 μl of the cDNA solution as a template according to the following schedule: 95° C. for 1 min, 55° C. for 1 min, and 72° C. for 1 min for 20 cycles. Among 83 samples tested, 57 samples show the expected size of bands, indicating expression of BMP4 these single cells. These samples are considered to be of allantoic mesodermal origin, and therefore excluded from amongst the candidates representing cells of PGC origin.

The expression of tissue non-specific alkaline phosphatase (TNAP), which has long been used as an early marker for PGCs (Ginsburg, M., Snow, M. H. L., and McLaren, A. (1990)), is then examined. Primer pairs are designed (5′: CCC AAA GCA CCT TAT TTT TCT ACC, 3′: TTG GCG AGT CTC TGC AAT TGG) and the same PCR reaction as above is performed. Amongst the 26 samples, 22 samples are judged to be positive for TNAP. From the alkaline phosphatase staining of the sectioned embryos, it is known that the somatic cells surrounding PGCs also express some amount of TNAP, although the level of expression is slightly lower than that in PGCs. Therefore, amongst these 22 positive samples there should be still be cells destined to become somatic cells as well as PGCs.

One of the genes known to be expressed in the totipotent PGCs but not in somatic cells is Oct4 (Yoem, Y. II., Fuhrmann, G., Ovitt, C. E., Brehm, A., Ohbo, K., Gross, M., Hubner, K., and Scholer, H. R. (1996)). To examine the possibility that Oct4 can be used as a marker to distinguish PGCs from somatic cells at this stage, Oct4 expression is checked in the 22 samples by PCR (5′: CAC TCT ACT CAG TCC CTT TTC, 3′: TGT GTC CCA GTC TTT ATT TAA G). All the 22 samples express Oct4 at comparable levels, indicating that the somatic cells at this stage are still actively transcribing Oct4 RNA.

The amount of expression of TNAP is quantitated in 22 samples by Southern blot analysis (reverse northern blot analysis). Given the fairly representative amplification of the single cell method, confirmed by amplifying single ES cell cDNA, Southern blot analysis allows semi-quantitative measurement of the amount of the genes expressed in the original single cells, although it does not serve as a perfect indicator of cell identity. However, as a result of this TNAP analysis, 10 samples out of 22 show relatively stronger bands at an equivalent level, while the remaining 12 samples exhibit weaker signals. These results indicate that these 22 samples can be divided at least into two groups, one with stronger TNAP expression (therefore from putative PGCs) and the other with weaker TNAP.

The possibility that somatic cells surrounding PGCs start to express Hoxb1, while PGCs do not (personal communication from Dr. Kirstie Lawson) is also examined. Primer pairs are designed (5′: AAC TCA TCA GAG GTC GAA GGA, 3′: CGG TGC TAT TGT AAG GTC TGC) and the same PCR reaction as above is performed. Among the 22 samples tested, 12 are positive, and more importantly, these 12 samples perfectly match the ones which show weaker TNAP signals, by Southern blot analysis.

Taking all these results into consideration, it is concluded that 10 samples out of 83, which are Oct4 (+), TNAP (++), BMP4 (−), and Hoxb1(−), are of PGC origin. This ratio (10/83) is reasonable, considering the number of the founding population of PGCs as 40 and the number of cells in the fragment as 250-300.

### Example 3

### Differential Screening of Single Cell cDNA Libraries

As the efficiency of the amplification of cDNA differs in each tube, it is very important to select the samples with the most efficiently amplified cDNA for the construction of libraries. The amplification of six different genes (ribosomal protein S12, intermediate filament protein vimentin, β tubulin-5, α actin, Oct4, E-cadherin) is examined in the 10 PGC candidate samples, by Southern blot analysis. Judging from the overall profile of the amplification of all these six genes, three cDNA preparations are selected for the construction of libraries.

To obtain the maximum amount of double strand cDNA, an extension step is performed with 5 μl of cell cDNA in 100 μl of the PCR buffer described as above (including 1 μl of Amplitaq) according to the following schedule: 94° C. for 5 min, 42° C. for 5 min, 72° C. for 30 min. The solution is extracted by phenol/chloroform treatment, and the amplified cDNAs are precipitated by ethanol, suspended in TE, and completely digested with EcoRI. The PCR primer and excess amount of dNTPs are removed by QIAGEN PCR Purification Kit, and all the purified cDNAs are run on a 2% low melting agarose gel. cDNAs above 500 bp are cut and purified by QIAGEN Gel Purification Kit. The purified cDNAs are precipitated by ethanol and suspended in TE and ligated into λ ZAP II vector arms. The ligated vector is packaged, titered and the ratio of the successfully ligated clones is monitored by amplifying the inserts with T3 and T7 primers from 20 plaques. More than 95% of the phage are found to contain inserts.

The representation of the three genes, ribosomal protein S12, β tubulin-5, Oct4, is quantitated by screening 5000 plaques, and the library of the best quality among the three (S12 0.62%, β tubulin 0.4%, Oct4 0.5%) is used for the differential screening. As a comparison partner with the PGC probe, one of the most efficiently amplified surrounding somatic cell cDNA (Oct4 (+), TNAP(+/−), BMP(−), and Hoxb1(+)) is selected by the similar Southern blot analysis.

The library is plated at a density of 1000 plaques per 15 cm dish to obtain large plaques (2 mm diameter) and two duplicate lifts are taken using Hybond N+filters from Amersham. The filters are prehybridized at 65° C. in 0.5M sodium phosphate buffer (pH7.3) containing 1% bovine serum albumin and 4% SDS. We prepared the cell cDNA probes by reamplifying for 10 cycles 1 μl of the original cell cDNA into 50 μl of total reaction with the AL1 primer, in the absence of cold dCTP and with 100 μCi of newly received ³²PdCTP, followed by the purification using Amersham Nick™ Spin Column. The filters are hybridised for at least 16 hrs with 1.0×10⁷ cpm/ml (The first filter is hybridised with somatic cell probe and the second filter is hybridised with the PGC probe). After the hybridisation, the filters are washed three times at 65° C. in 0.5×SSC, 0.5% SDS and exposed to X ray films until the appropriate signal is obtained (usually one to two days).

The positive plaques in the two duplicate filters are compared very carefully. Among 5000 plaques screened, 280 are picked as candidates representing the differentially expressed genes. The inserts of all the 280 plaques are amplified with T3 and T7 primers, run on 1.5% gels, and double sandwich Southern blotted. Each membrane is hybridised with the PGC and somatic cell probe, respectively, using the same conditions as the screening. 38 clones amongst the 280 are selected as differentially expressed genes. These clones are next hybridised with the second PGC and somatic cell cDNA probes, which resulted in 20 clones out of 38 to be common in both PGC cDNAs but they are either not included or less abundant in both somatic cell cDNAs. The sequences of all the 20 clones are determined.

Genes Highly Specific to the Earliest Population of PGCs

The 20 clones represent 11 different genes (two clones appear two times, one clone appears three times, and one clone appears 6 times). To further stringently check the specificity of expression, primer pairs are designed for these 11 clones and their expression checked in 10 different single PGC-candidate cDNAs and 10 different single somatic cell cDNAs by PCR. Two of them show highly specific expression to PGC cDNAs.

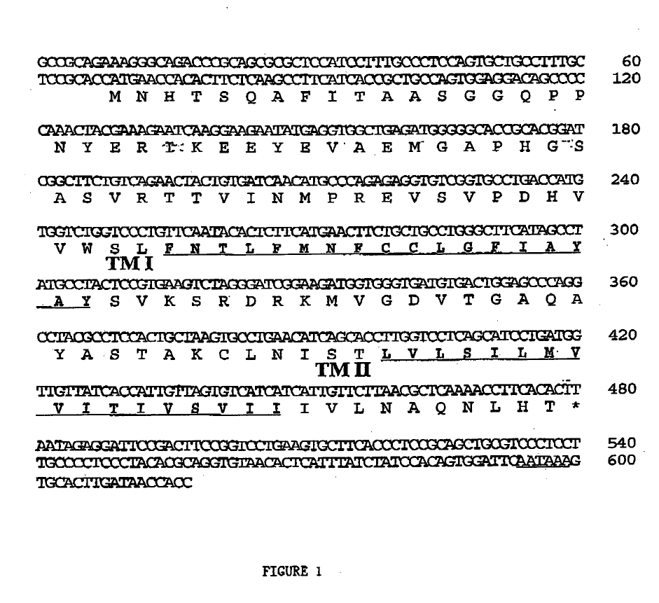

The first gene, GCR1 (Germ cell restricted-1, Fragilis), encodes a 137 amino acid protein with a predicted molecular weight of 15.0 kD. Nucleotide and amino acid sequences of mouse Fragilis are shown in FIG. 1.

The best fit model of the EMBL program PredictProtein predicts two transmembrane domains, both N and C terminus ends being located outside. The BLASP search revealed that Fragilis is a novel member of the interferon-inducible protein family. One prototype member, human 9-27 (identical to Leu-13 antigen), is inducible by interferon in leukocytes and endothelial cells, and is located at the cell surface as a component of a multimeric complex involved in the transduction of antiproliferative and homotypic adhesion signals (Deblandre, 1995). The BLASTN search revealed that the Fragilis sequence was found in ESTs derived from many different tissues both from embryos and adults, indicating that Fragilis may play a common role in different developmental and cell biological contexts. Database searches reveal a sequence match with the rat interferon-inducible protein (sp:INIB RAT, pir:JC1241) with unknown function. The GCR1 sequence appears six times in our screen, indicating high level expression in PGCs.

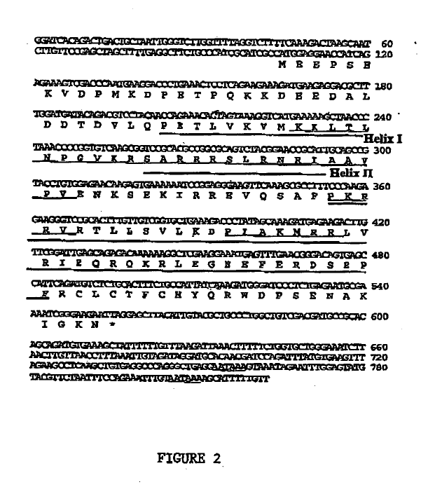

The second gene, GCR2, (Stella) encodes a 150 amino acid protein, of 18 kD. Nucleotide and amino acid sequences of mouse Fragilis are shown in FIG. 2.

It has no sequence homology with any known protein, contains several nuclear localisation consensus sequences and is highly basic pI (pI=9.67, the content of basic residues=23.3%), indicating a possible affinity to DNA. Furthermore a potential nuclear export signal was identified, indicating that Stella may shuttle between the nucleus and the cytoplasm. BLASTN analysis revealed that the Stella sequence was found only in the preimplantation embryo and germ line (newborn ovary, female 12.5 mesonephros and gonad etc.) ESTs indicating its predominant expression in totipotent and pluripotent cells. Interestingly, we found that Stella contains in its N terminus a modular domain which has some sequence similarity with the SAP motif. This motif is a putative DNA-binding domain involved in chromosomal orgainisation. Furthermore, the SMART program revealed the presence of a splicing factor motif-like structure in its C-terminus, These findings indicate a possible involvement of Stella in chromosomal orgainistion and RNA processing.

### Example 4

### Identification of PGCs by Screening for GCR1 and GCR2 Expression

Although PGCs are identified in Example 2 by analysis of BMP4, TNAP, Hoxb1, and Oct4, no single one of these genes can be taken as a marker for the PGC state. However, both GCR1 and GCR2 may be used as such.

The expression of GCR1 is examined. Primer pairs are designed (5′: CTACTCCGTGAAGTCTAGG, 3′: AATGAGTGTTACACCTGCGTG) and the same PCR reaction as above is performed. GCR1 expression was detected in germ cell competent cells. The definitive PGCs were recruited from amongst this group of cells showing expression of GCR1.

The boundary of GCR2 expression in particular is well-defined, and the expression is substantially limited to PGCs. Therefore, GCR2 is used as a positive marker for the selection of PGCs. Primer pairs are designed for amplifying the C terminal portion of GCR2 (5′: GCCATTCAGATGTCTCTGCAC, 3′: CTCACAGCTTGAGGCTTCTAA). The PCR amplification is performed using 0.5 μl of the cDNA solution obtained from PGCs in Example 1 as a template according to the following schedule: 95° C. for 1 min, 55° C. for 1 min, and 72° C. for 1 min for 20 cycles. Among 83 samples tested, only those taken from PGCs show expression of GCR2. Hence, GCR2 is a positive marker for the PGC fate.

Antibodies against GCR1 and GCR2 can be similarly used to detect pluripotent cells. Preferably, antibodies against GCR1 are used to detect germ cell competent cells, and antibodies against GCR2 are used to detect PGCs.

Accordingly, both GCR1 and GCR2 are positive markers for the PGC fate which can be used to positively identify PGC.

Identification of PGC by ISH

The in vivo expression of the two genes is examined by in situ hybridisation. The expression of GCR1 starts very weakly in the entire epiblast at E6.0-E6.5 (PreStreak stage) and becomes strong in the few cell layers of the proximal rim of the epiblast. BMP4 that is expressed in the extraembryonic ectoderm is one signalling molecule that is important for the induction of germ cell competence and expression of GCR1. Other signals, such as interferons are likely to be involved in the induction of GCR1. The expression becomes more intense at the proximo-posterior end of the developing primitive streak at the Early/Mid Streak stage and becomes very strong at this position from Late Streak stage onward. The expression persists until Early Head Fold stage and eventually disappears gradually. No expression is detected in the migrating PGCs at E8.5.

The expression of GCR2 starts at the proximo-posterior end of the developing primitive streak at Mid/Late Streak stage and becomes gradually strong at the same position from the later stage onward. The expression is specific and individual single cells stained in a dotted manner can be seen in the region where PGCs are considered to start differentiating as a cluster of cells. At Late Bud/Early Head Fold stage, some cells considered to be migrating from the initial cluster are stained as well as cells in the cluster. At E8.5 and E9.5, a group of cells considered to be the migrating PGCs are very specifically stained.

From these results, it is concluded that GCR1 is a gene which is upregulated during the process of lineage specification and germ cell competence, and subsequently of PGCs, when GCR2 is turned on after GCR1 to fix the PGC fate.

Accordingly, expression of GCR1 may be detected in a method of detecting lineage specification, and/or pluripotency, such as germ cell competence. Similarly, expression of GCR2 may be detected to detect commitment to cell fate, for example, commitment to fate as a primordial germ cell.

### Example 5

### Expression of Fragilis and Stella During Germ Line Development

Antibodies against Stella and Fragilis are used to detect expression of these genes in early embryos. It is found that each of these genes is expressed in primordial germ cells. In particular, we find that Fragilis is the first gene to mark PGC competent cells at the time of germ cell allocation. Stella is expressed only in the lineage-restricted founder PGCs and thereafter in the germ cell lineage.

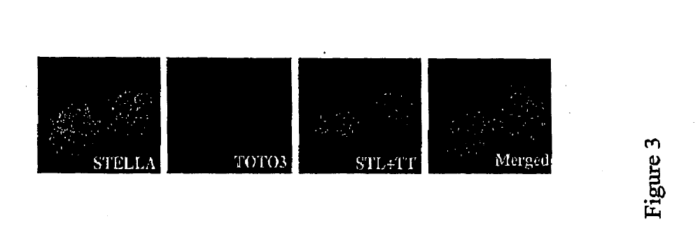

FIG. 3 shows expression of Fragilis in embryonic stem (ES) cells.

Fragilis is expressed in pluripotent ES and EG cells. During the derivation of EG cells from PGCs, it is found that Fragilis expression re-appears on EG cells. Late PGCs are negative for Fragilis after specification of these cells is completed.

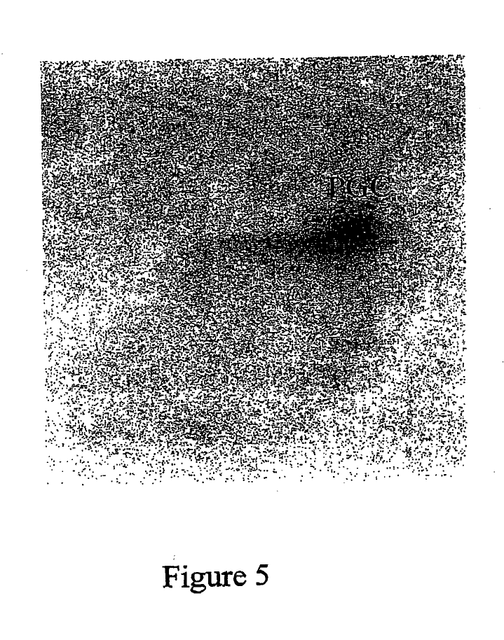

FIG. 5 shows expression of Fragilis as detected by whole-mount in situ hybridization in E7.2 mouse embryos.

There is strong Fragilis expression at the base of incipient allantois where the founder PGC population differentiates in the E7.25 embryos. Fragilis expression persisted until E7.5, but it was not detected in migrating PGCs at E8.5. Fragilis is first detected in germ cell competent proximal epiblast cells. Fragilis expression can be induced in the epiblast cells when combined with the tissues extraembryonic ectoderm tissues, which is the source of BMP4. In the BMP4 mutant mice, there is no expression of Fragilis, consistent with the absence of PGCs in these embryos (Lawson et al., 1999).

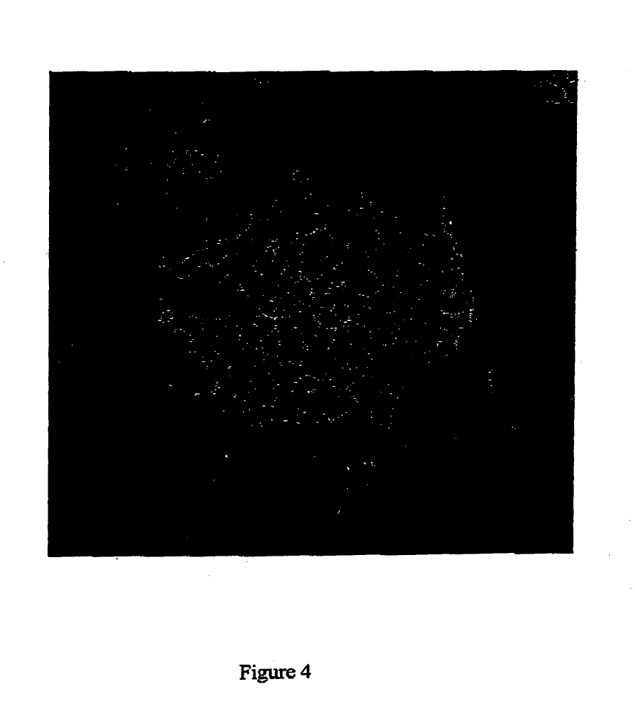

FIG. 4 shows expression of Stella in PGCs.

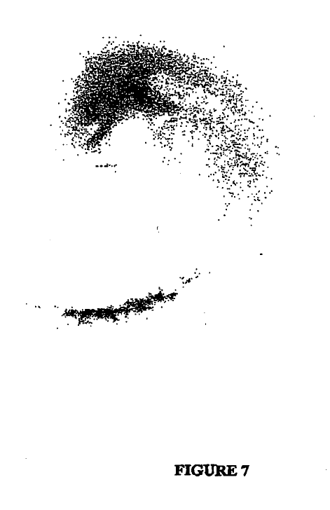

Stella expression which is strong in PGCs is downregulated in EG cells. There is also low level expression of Stella in ES cells. Stella and Fragilis are detectable in ES and EG cells by Northern blot analysis. Stella is first detected at E7.0 in single cells within the distinctive cluster of lineage-restricted PGCs, and thereafter in migrating PGCs and subsequently when they enter the gonads. FIG. 7 shows Stella expression in PGCs in the process of migration into the gonads in E9.0 embryos. Stella is the only gene so far known to be a definitive marker for the founder population of PGCs.

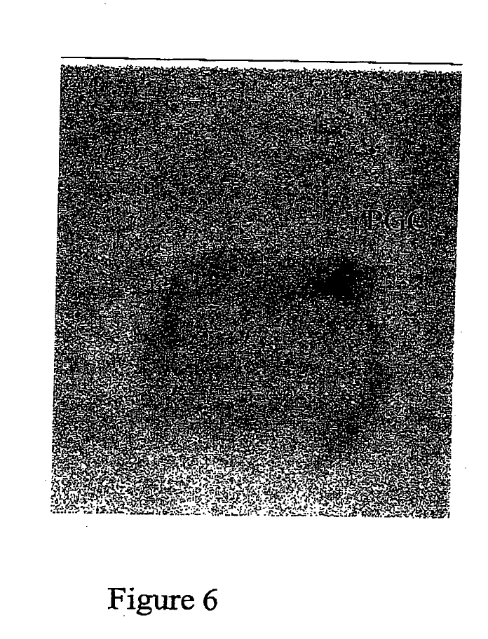

FIG. 6 shows expression of Stella as detected by whole-mount in situ hybridization in E7.2 mouse embryos.

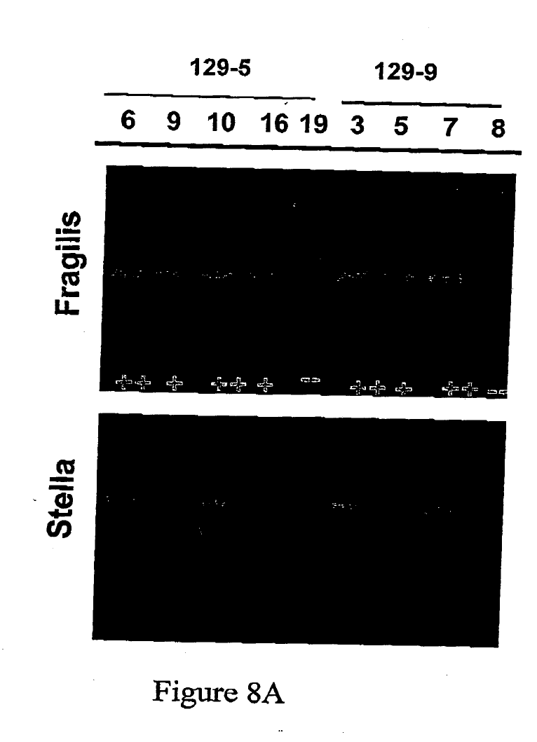

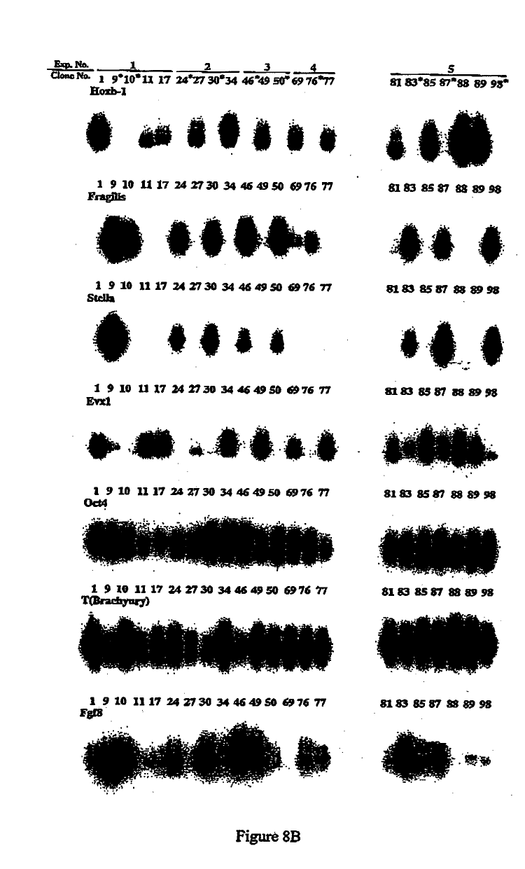

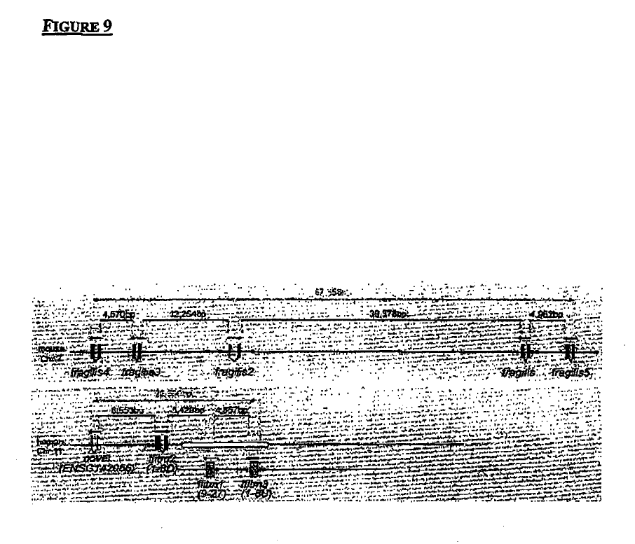

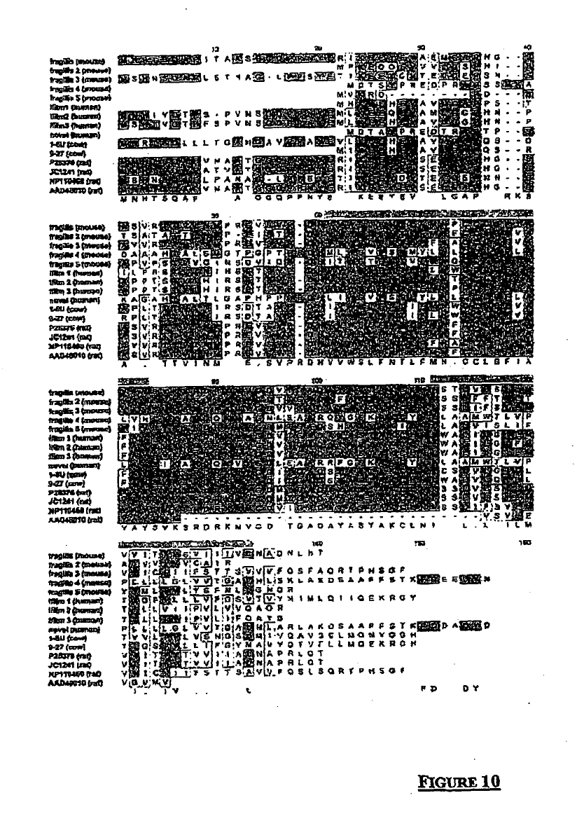

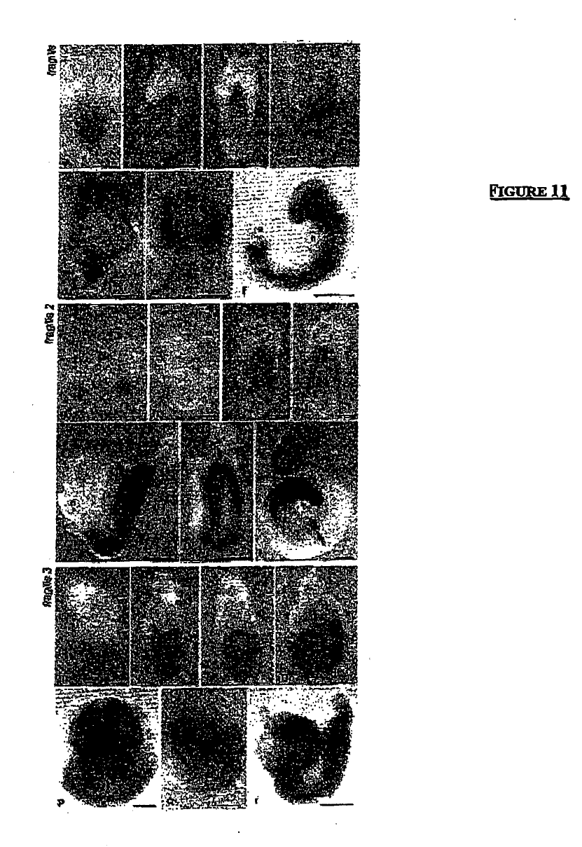

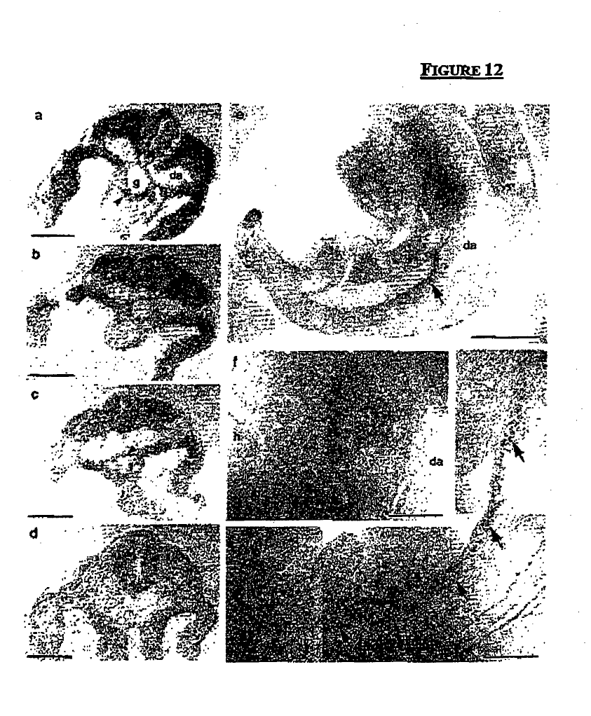

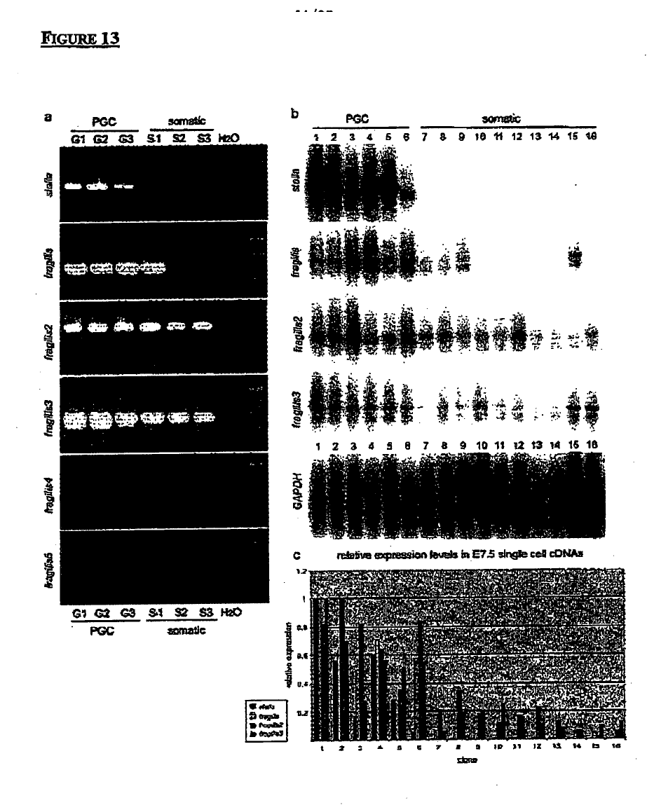

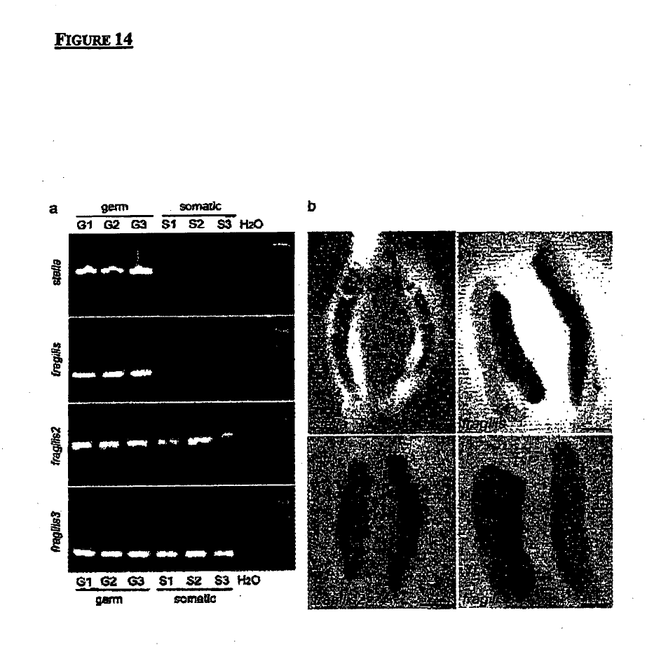

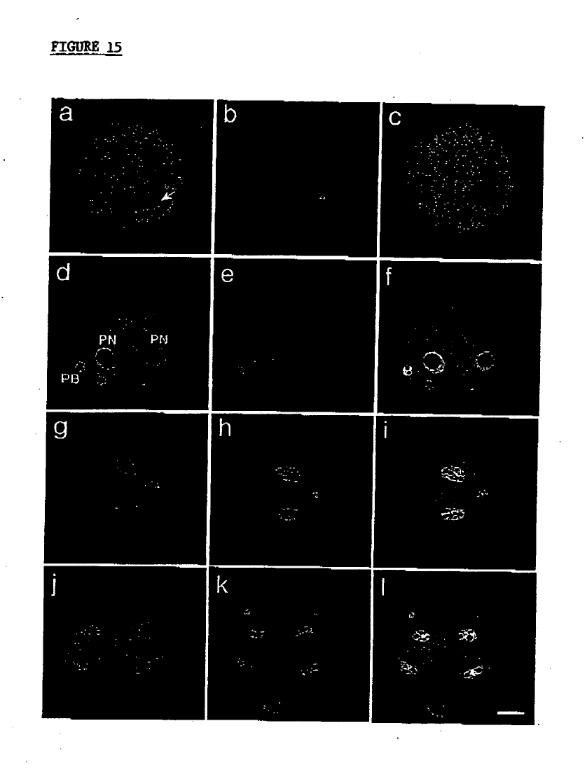

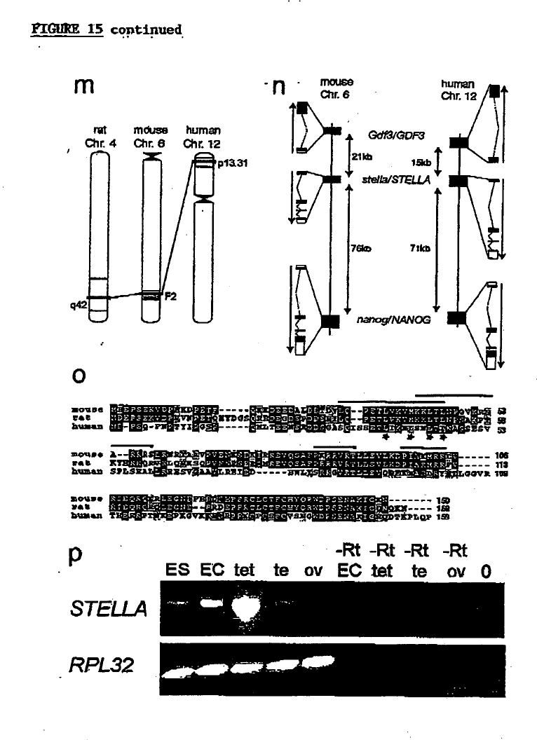

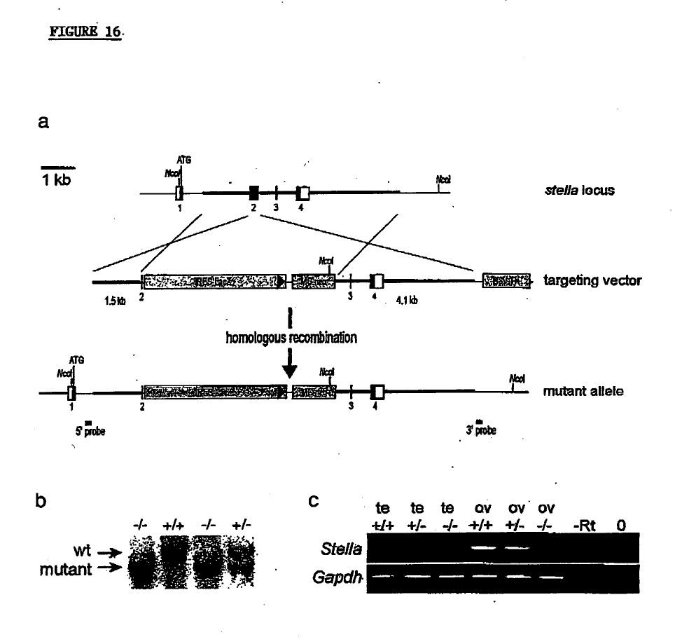

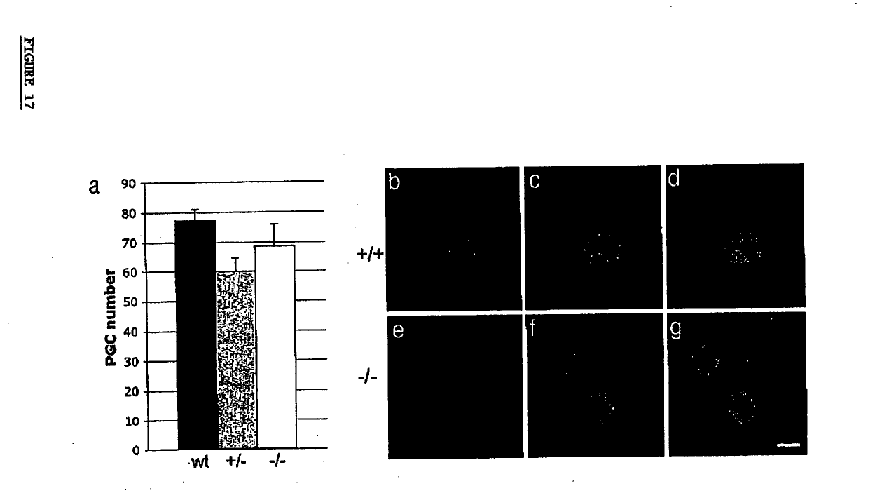

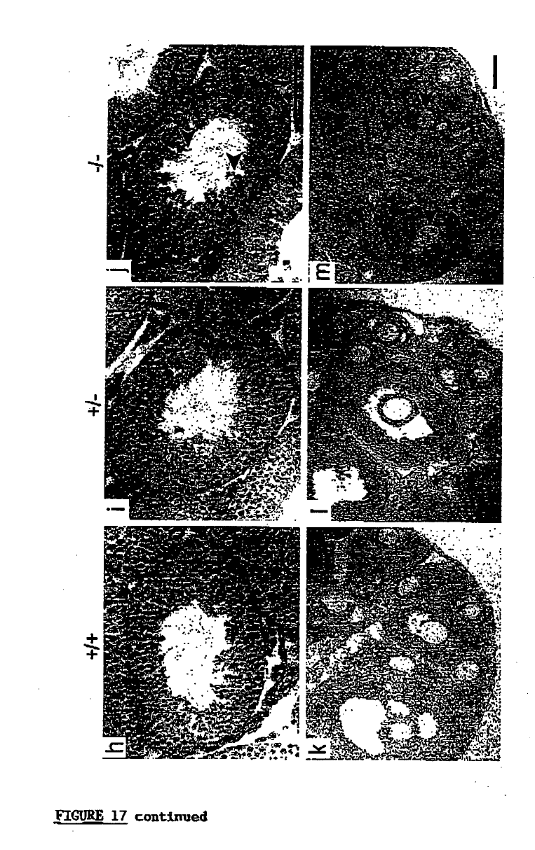

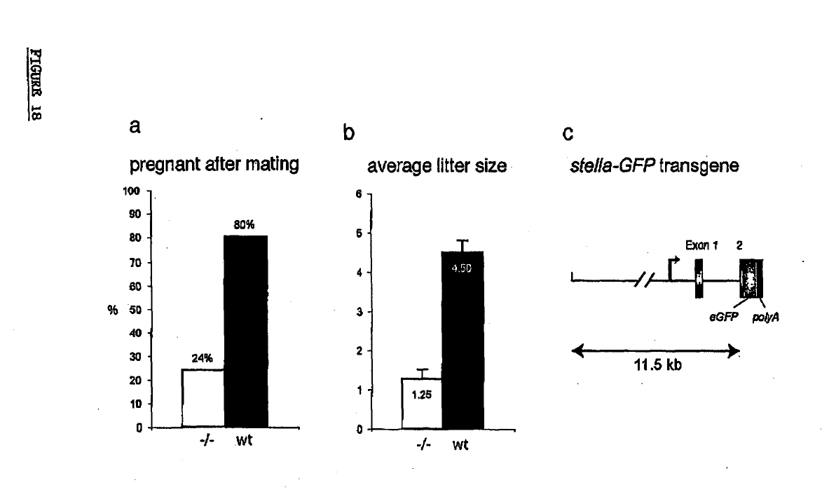

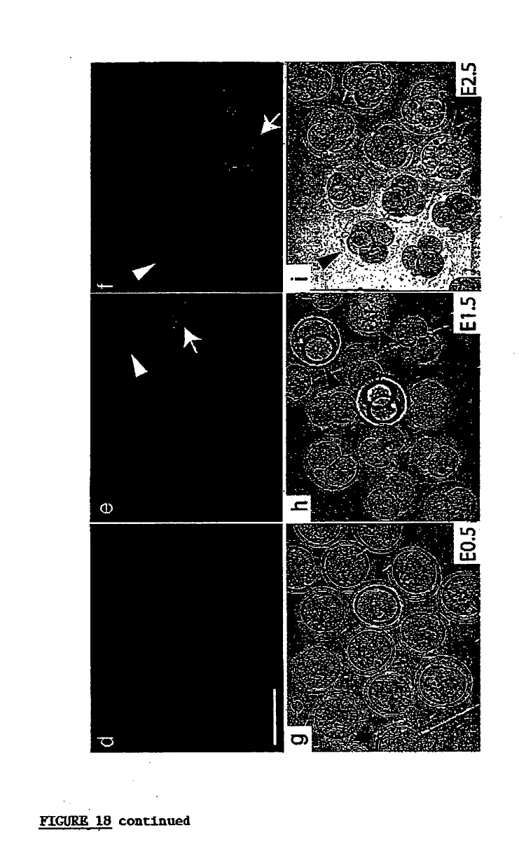

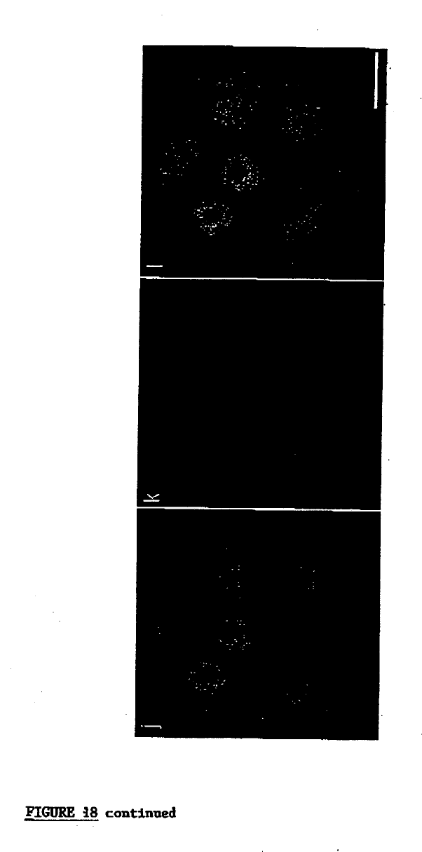

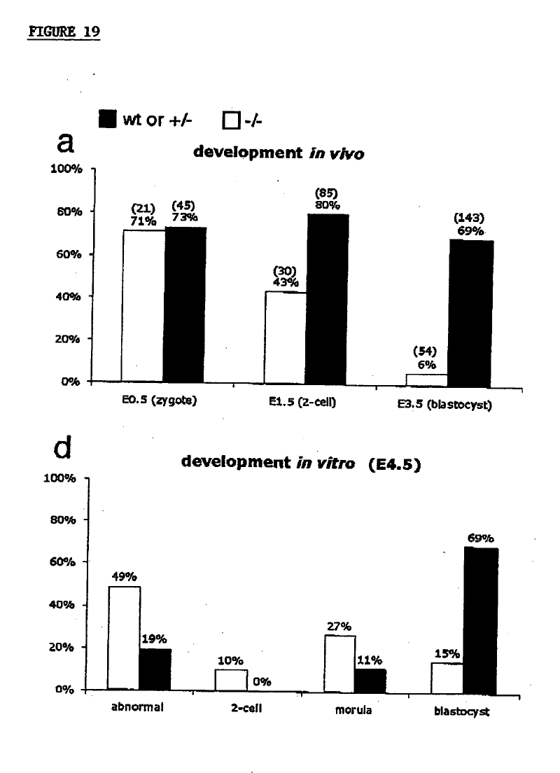

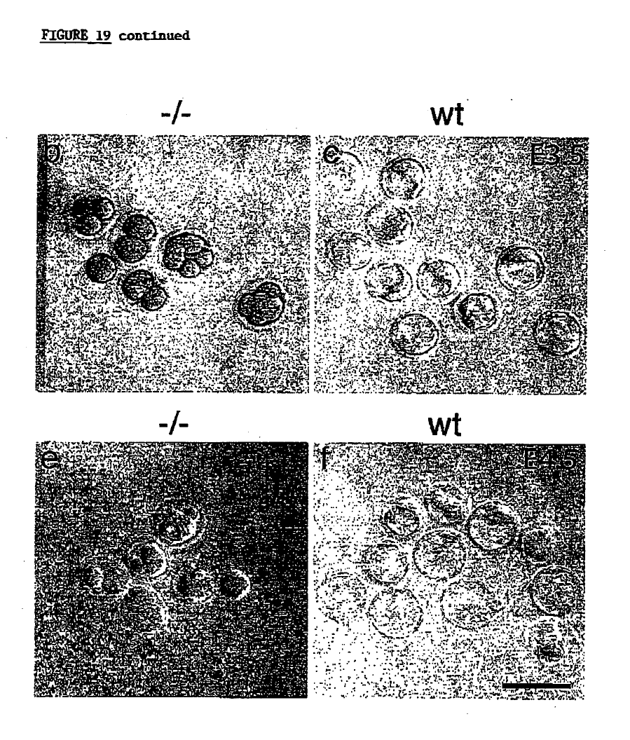

FIG. 8. Expression of Fragilis and Stella in single cells detected by PCR analysis of single cell cDNAs. Note that there are more single cells showing expression of Fragilis compared to those showing expression of Stella. Only cells with the highest levels of Fragilis expression are found to express Stella and acquire the germ cell fate. Cells that express Stella were found not to show expression of Hoxb1. Cells that express lower levels of Fragilis and no Stella become somatic cells and show expression of Hoxb1. The founder population of PGCs also show high levels of Tnap. Both the founder PGCs and the somatic cells show expression of Oct4, T(Brachyury), and Fgf8.

### Example 6

### Expression of Fragilis and Stella in Individual Cells

Intracellular localisation of Stella and Fragilis is also determined. Fragilis localised to a single cytoplasmic spot at the Golgi apparatus, as well as in the plasma membrane. Stella comprises a putative nuclear localisation signal and nuclear export signal, and is localised in both the cytoplasm and nucleus.

Fragilis is observed in the Golgi apparatus as well as in the plasma membrane of PGCs. The cell surface localization of Fragilis is expected as a member of the interferon inducible gene family [Deblandre, 1995]. Expression of Fragilis in the proximal rim of the epiblast marks the onset of germ cell competence. Fragilis has an IFN response element upstream of its exon 1, so it is very likely to be induced by IFN after initial priming by BMP4 of the proximal epiblast cells. These IFN inducible proteins can from a multimeric complex with other proteins such as TAPA1, which is capable of transduction of antiproliferative signals, which may be why the cell cycle time in founder PGCs increases from 6 to 16 hr, while the somatic cells continue to divide rapidly.

Stella, which has the putative nuclear localization signal and a nuclear export signal, was observed in both the cytoplasm and the nucleus. The onset of Stella is followed by the loss of Fragilis expression by E8.5. Therefore, Fragilis expression marks the onset of germ cell competence and Stella expression marks the end of this specification process. Expression of Stella in the founder PGCs marks an escape from the somatic cell fate and consistent with their pluripotent state. These studies indicate that specific set of genes are required to impose a germ line fate on cells that may otherwise become somatic cells. Stella, with its potential to shuttle between the nucleus and cytoplasm, could have a role in transcriptional and translational regulation, since many organisms possess elaborate transcriptional mechanisms to prevent germ cells from becoming somatic cells. Expression of Stella in the oocyte and preimplantation embryos indicates that it has a wider role in totipotency and pluripotency.

### Example 7

### The Link Between Fragilis and Stella

Only some of the cells that express Fragilis, ended up showing expression of Stella. Only those cells with the higest levels of Fragilis expression become PGCs and began to express Stella. Furthermore, Stella positive PGCs never show expression of Hoxb1. More importantly, only somatic cells with lower levels of Fragilis expression, show Hoxb1 expression. Furthermore, only the somatic cells show expression of two other homeobox-containing genes, Lim1 and Evx-1. Therefore lack of expression of Hoxb1, Evx-1 and Lim1, appears to be important for the specification of germ cell fate.

FIGS. 8a and 8b show expression of various genes in single cell PGCs and somatic cells by PCR analysis.

Our experiments also show that Oct4 is not a definitive marker of PGC, Previously, Oct4 expression is demonstrated in totipiotent and pluripotent cells [Nichols, 199, Pesce, 1998; Yeom, 1996]. However, we find that Oct4 is expressed to the same extent in all PGCs and somatic cells. We do however find expression of T (Brachyuri) and Fgf 8 in PGCs indicating that PGCs are recruited from amongst embryonic cells that are initially destined to become mesodermal cells.

### Example 8

### PGC Specification

The founder PGCs and their somatic neighbours share common origin from the proximal epiblast cells. By analysing the founder PGC and the somatic neighbour, a systematic screen for critical genes for the specification of germ cell fate has been established. Fragilis is an interferon (IFN) inducible gene that can promote germ cell competence and homotypic association to demarcate putative germ cells from their somatic neighbours, and such an example may apply to other situation during development. Expression of Stella occurs in cells with high expression of Fragilis. Fragilis is no longer required once germ cell specification is complete, but Stella expression continues in the germ cell lineage. Stella may also be important throughout in the totipotent/pluripotent cells since it is also expressed in oocytes and early preimplantion development embryos.

### Example 9

### Germ Line and Pluripotent Stem Cells

PGCs can be used to derive pluripotent embryonic germ (EG) cells. However, unlike EG cells, PGCs do not participate in development if introduced into blastocysts. They either cannot respond to signalling molecules, or that they are transcriptionally repressed. PGCs once specified do not express Fragilis on their cell surface. However, EG cells clearly show expression of Fragilis on their cell surface as do ES cells. Both EG and ES cells express Stella as judged by Northern analysis, although Stella is expressed at a lower level in ES and EG cells than in PGCs. Fragilis and Stella therefore have a role in pluripotent stem cells. These genes are therefore markers of these pluripotent stem cells, where they may also have a role in conferring pluripotency on these stem cells.

References

Brady, G. and Iscove, N. N. (1993). Construction of cDNA libraries from single cells. Methods Enzymol. 225, 611-623.

Dulac, C. and Axel, R. (1995). A novel family of genes encoding putative pheromone receptors in mammals. Cell 83, 195-206.

Ginsburg, M., Snow, M. H. L., and McLaren, A. (1990). Primordial germ cells in the mouse embryo during gastrulation. Development 110, 521-528.

Downs, K. M., and Davies, T. (1993). Staging of gastrulating mouse embryos by morphological landmarks in the dissecting microscope. Development 118, 1255-1266.

Lawson, K. A., Dunn, N. R., Roelen, B. A. J., Zeinstra, L. M., Davis, A. M., Wright, C. V. E., Korving, J. P. W. F. M., and Hogan, B. L. M. (1999). Bmp4 is required for the generation of primordial germ cells in the mouse embryo. Genes&Dev. 13, 424-436.

Yoem, Y. II., Fuhrmann, G., Ovitt, C. E., Brehm, A., Ohbo, K., Gross, M., Hubner, K., and Scholer, H. R. (1996). Germline regulatory element of Oct-4 specific for the totipotent cycle of embryonal cells. Development 1996, 881-894.

1. Weismann, A. Das Keimplasma. Eine theorie der Vereburg. Jenna Gustav Fischer (1892).

2. Eddy, E. M. Germ plasm and the differentiation of the germ cell line. Int Rev Cytol 43, 229-80 (1975).

3. Seydoux, G. & Strome, S. Launching the germline in Caenorhabditis elegans: regulation of gene expression in early germ cells. Development 126, 3275-83. (1999).

4. Wylie, C. Germ cells. Cell 96, 165-74. (1999).

5. Lawson, K. A. et al. Bmp4 is required for the generation of primordial germ cells in the mouse embryo. Genes Dev 13, 424-36. (1999).

6. Lawson, K. A. & Hage, W. J. Clonal analysis of the origin of primordial germ cells in the mouse. Ciba Found Symp 182, 68-84 (1994).

7. Tam, P. P. & Zhou, S. X. The allocation of epiblast cells to ectodermal and germ-line lineages is influenced by the position of the cells in the gastrulating mouse embryo. Dev Biol 178, 124-32. (1996).

8. Yoshimizu, T., Obinata, M. & Matsui, Y. Stage-specific tissue and cell interactions play key roles in mouse germ cell specification. Development 128, 481-90. (2001).

9. McLaren, A. Signaling for germ cells. Genes Dev 13, 373-6. (1999).

10. Ying, Y., Liu, X. M., Marble, A., Lawson, K. A. & Zhao, G. Q. Requirement of Bmp8b for the generation of primordial germ cells in the mouse. Mol Endocrinol 14, 1053-63. (2000).

11. Ying, Y., Qi, X. & Zhao, G. Q. Induction of primordial germ cells from murine epiblasts by synergistic action of BMP4 and BMP8B signaling pathways. Proc Natl Acad Sci USA 98, 7858-7862. (2001).

12. Ying, Y. & Zhao, G. Q. Cooperation of endoderm-derived BMP2 and extraembryonic ectoderm-derived BMP4 in primordial germ cell generation in the mouse. Dev Biol 232, 484-92. (2001).

13. Chiquoine, A. D. The identification, origin and migration of the primordial germ cells in the mouse embryo. Anat Rec 118,135-146 (1954).

14. Ginsburg, M., Snow, M. H. & McLaren, A. Primordial germ cells in the mouse embryo during gastrulation. Development 110, 521-8. (1990).

15. MacGregor, G. R., Zambrowicz, B. P. & Soriano, P. Tissue non-specific alkaline phosphatase is expressed in both embryonic and extraembryonic lineages during mouse embryogenesis but is not required for migration of primordial germ cells. Development 121, 1487-96. (1995).

16. Nichols, J. et al. Formation of pluripotent stem cells in the mammalian embryo depends on the POU transcription factor Oct4. Cell 95, 379-91. (1998).

17. Pesce, M., Gross, M. K. & Scholer, H. R. In line with our ancestors: Oct-4 and the mammalian germ. Bioessays 20, 722-32. (1998).

18. Yeom, Y. I. et al. Germline regulatory element of Oct-4 specific for the totipotent cycle of embryonal cells. Development 122, 881-94. (1996).

19. Downs, K. M. & Davies, T. Staging of gastrulating mouse embryos by morphological landmarks in the dissecting microscope. Development 118, 1255-66. (1993).

20. Brady, G. & Iscove, N. N. Construction of cDNA libraries from single cells. Methods Enzymol 225, 611-23 (1993).

21. Dulac, C. & Axel, R. A novel family of genes encoding putative pheromone receptors in mammals. Cell 83, 195-206. (1995).

22. Frohman, M. A., Boyle, M. & Martin, G. R. Isolation of the mouse Hox-2.9 gene; analysis of embryonic expression suggests that positional information along the anterior-posterior axis is specified by mesoderm. Development 110, 589-607. (1990).

23. Deblandre, G. A. et al. Expression cloning of an interferon-inducible 17-kDa membrane protein implicated in the control of cell growth. J Biol Chem 270, 23860-6. (1995).

24. Friedman, R. L., Manly, S. P., McMahon, M., Kerr, I. M. & Stark, G. R. Transcriptional and posttranscriptional regulation of interferon-induced gene expression in human cells. Cell 38, 745-55. (1984).

25. Evans, S. S., Collea, R. P., Leasure, J. A. & Lee, D. B. IFN-alpha induces homotypic adhesion and Leu-13 expression in human B lymphoid cells. J Immunol 150, 736-47. (1993).

26. Evans, S. S., Lee, D. B., Han, T., Tomasi, T. B. & Evans, R. L. Monoclonal antibody to the interferon-inducible protein Leu-13 triggers aggregation and inhibits proliferation of leukemic B cells. Blood 76, 2583-93. (1990).

27. Aravind, L. & Koonin, E. V. SAP—a putative DNA-binding motif involved in chromosomal organization. Trends Biochem Sci 25, 112-4. (2000).

28. Gurdon, J. B., Lemaire, P. & Kato, K. Community effects and related phenomena in development. Cell 75, 831-4. (1993).

29. Reid, L. E. et al. A single DNA response element can confer inducibility by both alpha- and gamma-interferons. Proc Natl Acad Sci USA 86, 840-4. (1989).

30. Kita, M. et al. [Expression of cytokines and interferon-related genes in the mouse embryo]. C R Seances Soc Biol Fil 188, 593-600 (1994).

31. Gomperts, M., Garcia-Castro, M., Wylie, C. & Heasman, J. Interactions between primordial germ cells play a role in their migration in mouse embryos. Development 120, 135-41. (1994).

32. Herrmann, B. G., Labeit, S., Poustka, A., King, T. R. & Lehrach, H. Cloning of the T gene required in mesoderm formation in the mouse. Nature 343, 617-22. (1990).

33. Herrmann, B. G. Expression pattern of the Brachyury gene in whole-mount TWis/TWis mutant embryos. Development 113, 913-17

34. Crossley, P. H. & Martin, G. R. The mouse Fgf8 gene encodes a family of polypeptides and is expressed in regions that direct outgrowth and patterning in the developing embryo. Development 121,439-51. (1995).

35. Barnes, J. D., Crosby, J. L., Jones, C. M., Wright, C. V. & Hogan, B. L. Embryonic expression of Lim-1, the mouse homolog of Xenopus Xlim-1, suggests a role in lateral mesoderm differentiation and neurogenesis. Dev Biol 161, 168-78. (1994).

36. Fujii, T. et al. Expression patterns of the murine LIM class homeobox gene lim1 in the developing brain and excretory system. Dev Dyn 199, 73-83. (1994).

37. Bastian, H. & Gruss, P. A murine even-skipped homologue, Evx 1, is expressed during early embryogenesis and neurogenesis in a biphasic manner. Embo J 9, 1839-52. (1990).

38. Rogers, M. B., Hosler, B. A. & Gudas, L. J. Specific expression of a retinoic acid-regulated, zinc-finger gene, Rex-1, in preimplantation embryos, trophoblast and spermatocytes. Development 113, 815-24. (1991).

39. Sutton, J. et al. Genesis, a winged helix transcriptional repressor with expression restricted to embryonic stem cells. J Biol Chem 271, 23126-33. (1996).

40. Cox, D. N. et al. A novel class of evolutionarily conserved genes defined by piwi are essential for stem cell self-renewal. Genes Dev 12, 3715-27. (1998).

41. Fujiwara, Y. et al. Isolation of a DEAD-family protein gene that encodes a murine homolog of Drosophila vasa and its specific expression in germ cell lineage. Proc Natl Acad Sci USA 91, 12258-62. (1994).

42. Dixon, K. E. Evolutionary aspects of primordial germ cell formation. Ciba Found Symp 182, 92-110 (1994).

43. Mahowald, A. P. Assembly of the Drosophila germ plasm. Int Rev Cytol 203, 187-213 (2001).

44. Nieuwkoop, P. D. & Satasurya, L. A. Primordial germ cells in the chordates. Cambridge University Press, Cambridge, UK (1979).

45. Johnson, A. D., Bachvarova, R. F., Drum, M. & Masi, T. Expression of axolotl dazl rna, a marker of germ plasm: widespread maternal ma and onset of expression in germ cells approaching the gonad. Dev Biol 234, 402-15. (2001).

46. Johnson, A. D., Bachvarova, R. F., Masi, T. & Drum, M. Expression of Vasa and Daz-like genes demonstrate that Axolotl primordial germ cells (PGCs) are not predetermined. Germ cells Cold Spring harbor laboratory, 61 (2000).

47. Toyooka, Y. et al. Expression and intracellular localization of mouse Vasa-homologue protein during germ cell development. Mech Dev 93, 139-49. (2000).

48. Saitou, M. et al. Occludin-deficient embryonic stem cells can differentiate into polarized epithelial cells bearing tight junctions. J Cell Biol 141, 397-408. (1998).

49. Henrique, D. et al. Expression of a Delta homologue in prospective neurons in the chick. Nature 375, 787-90. (1995).

50. Wilkinson, D. G. & Nieto, M. A. Detection of messenger RNA by in situ hybridization to tissue sections and whole mounts. Methods Enzymol 225, 361-73 (1993).

51. Winnier, G., Blessing, M., Labosky, P. A. & Hogan, B. L. Bone morphogenetic protein-4 is required for mesoderm formation and patterning in the mouse. Genes Dev 9, 2105-16. (1995).

Each of the applications and patents mentioned in this document, and each document cited or referenced in each of the above applications and patents, including during the prosecution of each of the applications and patents (“application cited documents”) and any manufacturer's instructions or catalogues for any products cited or mentioned in each of the applications and patents and in any of the application cited documents, are hereby incorporated herein by reference. Furthermore, all documents cited in this text, and all documents cited or referenced in documents cited in this text, and any manufacturer's instructions or catalogues for any products cited or mentioned in this text, are hereby incorporated herein by reference.

Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the claims.

1. A GCR1 polypeptide or a GCR2 polypeptide, or a fragment, homologue, variant or derivative thereof.

2. A polypeptide according to claim 1, which has at least 50% homology to a sequence shown in SEQ ID NO: 2.

3. The polypeptide of claim 2, which has at least 60% homology to a sequence shown in SEQ ID NO: 2.

4. The polypeptide of claim 3, which has at least 70% homology to a sequence shown in SEQ ID NO: 2.

5. The polypeptide of claim 4, which has at least 80% homology to a sequence shown in SEQ ID NO: 2.

6. The polypeptide of claim 5, which has at least 90% homology to a sequence shown in SEQ ID NO: 2.

7. The polypeptide of claim 6, which has at least 95% homology to a sequence shown in SEQ ID NO: 2.

8. A nucleic acid encoding a polypeptide according to claim 1, or a fragment, homologue, variant or derivative thereof.

9. A nucleic acid comprising a sequence of 25 contiguous nucleotides of the nucleic acid of claim 8.

10. A nucleic acid comprising a sequence of 15 contiguous nucleotides of the nucleic acid of claim 8.

11. A nucleic acid having at least 90% homology with the sequence set forth in SEQ ID NO: 1, or a fragment, variant or derivative thereof.

12. A nucleic acid comprising a sequence of 25 contiguous nucleotides of the nucleic acid of claim 11.

13. A nucleic acid comprising a sequence of 15 contiguous nucleotides of the nucleic acid of claim 11.

14. A nucleic acid having at least 75% homology with the sequence set forth in SEQ ID NO: 3, SEQ ID NO: 5, SEQ ID NO: 6, SEQ ID NO: 7, SEQ ID NO: 8 or SEQ ID NO: 9, or a fragment, variant or derivative thereof.

15. A nucleic acid comprising a sequence of 25 contiguous nucleotides of the nucleic acid according to claim 14.

16. A nucleic acid comprising a sequence of 15 contiguous nucleotides of the nucleic acid according to claim 14.

17. The complement of a nucleic acid sequence according to claim 8.

18. The complement of a nucleic acid sequence according to claim 11.

19. The complement of a nucleic acid sequence according to claim 14.

20. A nucleic acid according to claim 8, comprising one or more nucleotide substitutions, wherein such substitutions do not alter the coding specificity of said nucleic acid as a result of the degeneracy of the genetic code.

21. A nucleic acid according to claim 11, comprising one or more nucleotide substitutions, wherein such substitutions do not alter the coding specificity of said nucleic acid as a result of the degeneracy of the genetic code.

22. A nucleic acid according to claim 14, comprising one or more nucleotide substitutions, wherein such substitutions do not alter the coding specificity of said nucleic acid as a result of the degeneracy of the genetic code.

23. A polypeptide encoded by a nucleic acid according to claim 8.

24. A polypeptide encoded by a nucleic acid according to claim 11.

25. A polypeptide encoded by a nucleic acid according to claim 14.

26. A polypeptide according to claim 23, in which the polypeptide comprises a sequence shown in SEQ ID NO: 2 or SEQ ID NO: 4.

27. A polypeptide according to claim 24, in which the polypeptide comprises a sequence shown in SEQ ID NO: 2 or SEQ ID NO: 4.

28. A polypeptide according to claim 25, in which the polypeptide comprises a sequence shown in SEQ ID NO: 2 or SEQ ID NO: 4.

29. A method for identifying a pluripotent cell, comprising detecting the presence of a polypeptide according to claim 1 or a polypeptide encoded by a nucleic acid encoding a polypeptide according to claim 1, or a fragment, homologue, variant or derivative thereof, or the expression of a nucleic acid encoding a polypeptide according to claim 1, or a fragment, homologue, variant, homologue, or derivative thereof, or a nucleic acid having at least 90% homology with the sequence set forth in SEQ ID NO: 1, or a fragment, variant, homologue or derivative thereof, or a nucleic acid having at least 75% homology with the sequence set forth in SEQ ID NO: 3, SEQ ID NO: 5, SEQ ID NO: 6, SEQ ID NO: 7, SEQ ID NO: 8 or SEQ ID NO: 9, or a fragment, variant, homologue or derivative thereof.

30. A method according to claim 29, comprising the steps of amplifying nucleic acids from a putative pluripotent cell using 5′ and 3′ primers specific for GCR1 and/or GCR2, and detecting amplified nucleic acid thus produced.

31. A method according to claim 29, wherein the expression of the nucleic acid sequence is detected by in situ hybridisation.

32. A method according to claim 15, wherein the expression of the nucleic acid sequence is determined by detecting the protein product encoded thereby.

33. A method according to claim 29, wherein the protein product is detected by immunostaining.

34. A method according to claim 32, wherein the protein product is detected by immunostaining.

35. An antibody specific for a polypeptide according to claim 1, a polypeptide encoded by a nucleic acid encoding a polypeptide according to claim 1, or a fragment, homologue, variant or derivative thereof.

36. An antibody according to claim 35, which is capable of specifically binding to an extracellular domain of GCR1.

37. A method of using an antibody according to claim 35 for the identification and/or isolation of a pluripotent cell.

38. A pluripotent cell identified by a method comprising detecting the presence of a polypeptide according to claim 1 or a polypeptide encoded by a nucleic acid encoding a GCR1 polypeptide or a GCR2 polypeptide, or a fragment, homologue, variant or derivative thereof, or a fragment, homologue, variant or derivative thereof, or the expression of a nucleic acid encoding a GCR1 polypeptide or a GCR2 polypeptide, or a fragment, homologue, variant or derivative thereof, or a fragment, homologue, variant, homologue, or derivative thereof, or a nucleic acid having at least 90% homology with the sequence set forth in SEQ ID NO: 1, or a fragment, variant, homologue or derivative thereof, or a nucleic acid having at least 75% homology with the sequence set forth in SEQ ID NO: 3, SEQ ID NO: 5, SEQ ID NO: 6, SEQ ID NO: 7, SEQ ID NO: 8 or SEQ ID NO: 9, or a fragment, variant, homologue or derivative thereof.

39. A pluripotent cell identified by a method of using an antibody according to claim 35 for the identification and/or isolation of a pluripotent cell.

40. A method for isolating a gene specifically expressed in a pluripotent cell, comprising the steps of (a) providing a population of cells containing a pluripotent cell; (b) isolating one or more pluripotent cells therefrom and providing single-cell pluripotent cell isolates; (c) amplifying the transcribed nucleic acid present in a single pluripotent cell; (d) conducting a subtractive hybridisation screen to identify transcripts present in pluripotent cells but not in somatic cells; and (e) probing a nucleic acid library with one or more transcripts identified in (d) to clone one or more genes which are specifically expressed in pluripotent cells.

41. A method according to claim 29, in which the pluripotent cell is selected from the group consisting of a primordial germ cell (PGC), an embryonic stem cell (ES) and an embryonic germ cell (EG).

42. A method according to claim 33, in which the pluripotent cell is selected from the group consisting of a primordial germ cell (PGC), an embryonic stem cell (ES) and an embryonic germ cell (EG).

43. A method according to claim 37, in which the pluripotent cell is selected from the group consisting of a primordial germ cell (PGC), an embryonic stem cell (ES) and an embryonic germ cell (EG).

44. A method according to claim 40, in which the pluripotent cell is selected from the group consisting of a primordial germ cell (PGC), an embryonic stem cell (ES) and an embryonic germ cell (EG).

45. A pluripotent cell according to claim 40, in which the pluripotent cell is selected from the group consisting of a primordial germ cell (PGC), an embryonic stem cell (ES) and an embryonic germ cell (EG).
